(19) United States
(12) Patent Application Publication
(10) Pub. No.: US 2009/0043109 A1
(43) Pub. Date: Feb. 12, 2009

(54) ASYMMETRIC ONE- AND TWO-PHOTON FLUOROPHORES FOR SIMULTANEOUS DETECTION OF MULTIPLE ANALYTES USING A COMMON EXCITATION SOURCE

(76) Inventors: Brenda D. Spangler, Livingston, MT (US); Charles W. Spangler, Livingston, MT (US); E. Scott Tarter, Bozeman, MT (US)

Correspondence Address: Antoinette M. Tease, P.L.L.C., PO Box 51016, Billings, MT, 59105, US

(21) Appl. No.: 11/834,683

(22) Filed: Aug. 7, 2007

(51) Int. Cl. C07D 209/82; C07C 59/11

(52) U.S. Cl. 548/446; 562/465

(57) ABSTRACT

A family of one- and two-photon dyes that have overlapping fluorescence excitation maxima between 365 nm and 425 nm and that are specifically functionalized for coupling to biomolecules. The dyes have defined Stokes shifts such that they produce a continuum of emission wavelengths. The dyes are asymmetrical bis stilbenes. The asymmetry of these dyes facilitates functionalization of the dyes with carboxyl, thiol, aldehyde-reactive or amine-reactive groups, which in turn facilitates coupling with macromolecules. Furthermore, the dyes of the present invention are photostable and have high quantum yields (fluorescence intensity).

### STATEMENT REGARDING FEDERALLY SPONSORED RESEARCH OR DEVELOPMENT

This invention was made with Government support under Award Nos. DMI-0318842 and DMI-0450469 awarded by the National Science Foundation. The Government has certain rights in this invention.

### BACKGROUND OF THE INVENTION

1. Field of the Invention

The present invention relates generally to the field of fluorescent dyes, and more specifically, to asymmetric one- and two-photon dyes that have overlapping fluorescence excitation maxima between 365 nm and 425 nm and that are specifically functionalized for coupling to biomolecules.

2. Description of the Related Art

Fluorescent dye-labeled polymeric nanoparticles or microspheres, dextrans, antibodies and ligands have been used as biomarkers and labels in various types of assays including lateral flow diffusion assays, and for immunodetection or ligand-based tracking. The particles are typically infused with dye, or the dyes can be directly conjugated to the surface of functionalized particles for use in lateral diffusion assays (see, e.g., U.S. Patent Application Pub. Nos. 20040101822 (Wiesner et al.) and 20050058601 (Liu)), or the dyes are conjugated to biomarkers for use in tracking cellular components in vivo, for example, using a fluorescent-labeled antibody that recognizes a specific cell-surface component then exposing cells in culture to the conjugate and observing localization of the antibody-antigen complex with a fluorescence microscope. Fluorescent-labeled ligands can be internalized into cells and can each bind their target analyte, thereby localizing its position within a cell.

Use of multiple ligands, each specifically labeled with dyes that are distinguishable from one another, is limited by the fact that most dyes have different excitation maxima, necessitating multiple excitation sources and filters. For this reason, only a limited number of ligands—typically only rhodamine and fluorescein, which have similar excitation maxima—can be used simultaneously to image cell components. It would be advantageous to be able to image three or more different cellular features simultaneously, particularly in vivo, and to observe multiple potential interactions between intracellular organelles or metabolites.

In lateral diffusion assays, only single analytes can be detected on a single test strip if gold nanoparticles are used as carriers for probe ligands because gold nanoparticles captured on a membrane by a band of immobilized capture ligand will fluoresce pink only. Thus, only a single test signal (pink band) is possible in addition to the control line unless the strip has a multiple addressing (positional) readout that can be specified. Most lateral diffusion assays do not have this option. It is difficult, if not impossible, to modify the color of a gold nanoparticle readout.

Latex or polystyrene beads can be coated or infiltrated with colored dyes and mixed to provide a cocktail of colors, but the number of suitable dyes is limited, and the sensitivity is generally less than gold nanoparticle carriers. Fluorescent beads of this type are available commercially from several sources, one of which is Bangs Laboratories, Inc. of Fishers, Ind. Fluorescent dyes provide a much more sensitive readout, but only a few fluorescent dyes have a common excitation, making visualization of multiple analytes simultaneously on a single test strip difficult or impossible. Because dyes often have dissimilar structural and optical characteristics, optimization of multiplex assays becomes very complex and time consuming. Single strip assays for identification of a specific strain of pathogen in a clinical sample, for example, or for identification of the many potential pathogens in a stool sample or nasal swab, would be facilitated by a cocktail of dye-labeled ligands to be used with a single clinical sample and read simultaneously using a single excitation source.

Multiplexed detection is commonly practiced in commercial real-time polymerase chain reaction (PCR) assays and electrophoretic assays (see U.S. Patent Application Pub. No. 20020197649 (Singh)). These types of assays are often limited by the number of filters necessary for multiple excitation wavelengths and emission wavelengths. Structurally and chemically similar fluorophores capable of excitation with a single wavelength source would represent a unique set of spectrally distinguishable dyes for multiplex applications.

Luminescence is the emission of light from any substance, and it occurs from electronically excited states. Luminescence can be divided into two categories. The first category is phosphorescence, in which light, in the form of photons, is absorbed but is emitted slowly from an electronically excited state whose transition back to the ground state is quantum mechanically forbidden. Phosphorescence is characterized by long, slow emissions and even “glow-in-the dark” phenomena.

The other category is fluorescence. One-photon fluorescence typically occurs from aromatic molecules when light in the form of photons is absorbed and produces electronically excited states whose rapid return to the ground state by emission of a photon is quantum mechanically allowed. It is also possible for a molecule to simultaneously absorb two or more long-wavelength photons, generated by a high-intensity pulsed laser. The excited singlet state achieved in this process is determined by the symmetry of the molecule. Emission, as with one-photon absorption, occurs rapidly at a fixed wavelength, longer than the absorption wavelength due to relaxation phenomena that returns the electron to the lowest excited singlet state.

The difference between excitation wavelength and emission wavelength is called the Stokes shift. The dyes of the present invention are capable of both one-photon and two-photon absorption and subsequent emission at longer wavelengths. In the applications using excitation wavelengths between 350 nm and 550 nm, the dyes act as one-photon absorbing fluorophores. When they are excited by wavelengths above 600 nm with pulsed laser irradiation, for example, as in confocal microscopy, they behave as two-photon absorbing fluorophores.

Chemical structures based on distyrylbenzene have been used in optoelectronic applications (see U.S. Pat. No. 7,094,929 (Bazan et alt, 2006), and symmetrical bis stilbenes with and without diphenylamine substituents have been patented or are patent pending as structures for multiphoton optical materials (see U.S. Pat. No. 6,267,913 (Marder et al., 2001) and U.S. Patent Application Pub. No. 20020185634 (Marder et al.)). Such structures have not been reduced to practice in biological applications, however.

The two-photon cross-sections of rhodamine and fluorescein (the most frequently used dyes for two-photon applications) are quite low (200-300 Goeppert-Meyer (GM) units and 10 GM units, respectively) compared to the two-photon cross-sections ranging from 100 GM to 1,000 GM for the dyes described herein. Large two-photon cross-sections allow enhanced three-dimensional spatial control.

Remarkable advances have taken place in imaging at the cellular level using two-photon absorption due to the fact that two-photon absorption is inherently a 3D event, i.e., two-photon fluorescence is only observed at the focal point of the focused laser beam. One-photon fluorophores do not have this spatial resolution capability. This capability allows highly detailed imaging of specific regions of a living cell without attending background fluorescence. Although this fact has been known for some time, it has only become practical with the availability of fluorophores that exhibit extremely large intrinsic two-photon cross-sections that can be bio-conjugated to specific molecular probes. Two-photon fluorophores that contain functionalities that permit conjugation to a wide variety of biomolecules provide a means of enhancing deep-tissue diagnostics and potential detection of deep-seated bacterial infections.

Existing fluorescent dye-labeled nanoparticles may be of varying brightness, particularly because they are excited at less than optimal wavelengths. A family of fluorescent dyes with similar quantum yields when excited with a single wavelength source conjugated to specific ligands would improve detection and analysis of multiple target analytes. Quantum dots, either alone or encased in ceramic or polystyrene microspheres, fulfill this objective, but they are composed of toxic materials such as cadmium, tellurium and selenium, that make large-scale production of quantum dot particles for assay difficult in terms of hazardous waste disposal, and in vivo applications unfeasible.

Accordingly, it is an object of the present invention to provide physiologically acceptable dyes that can be used to image three or more different cellular features simultaneously, particularly in vivo, and to observe multiple potential interactions between intracellular organelles or metabolites. It is a further object of the present invention to provide structurally and chemically similar fluorophores capable of excitation with a single wavelength source, which would represent a unique set of spectrally distinguishable dyes for multiplex applications. It is yet another object of the present invention to provide a family of fluorescent dyes with similar quantum yields when excited with a single wavelength source conjugated to specific ligands so as to improve detection and analysis of multiple target analytes.

It is a further object of the present invention to provide dyes that facilitate the optimization of multiplex assays, for example, dyes that could be used in single strip assays for identification of a specific strain of pathogen in a clinical sample or for identification of the many potential pathogens in a stool sample or nasal swab. It is yet another object of the present invention to provide the ability to use a cocktail of dye-labeled ligands in a single clinical sample and to read those ligands simultaneously using a single excitation source.

It is yet another object of the present invention to provide two-photon dyes with higher cross-sections for biological applications, for example, confocal microscopy.

### BRIEF SUMMARY OF THE INVENTION

In one embodiment, the present invention is a fluorophore having the following chemical structure:

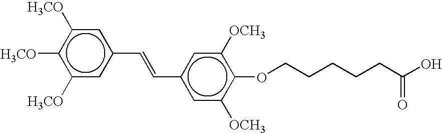

In an alternate embodiment, the present invention is a fluorophore having the following chemical structure:

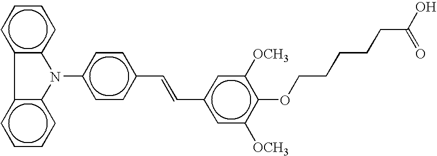

In an alternate embodiment, the present invention is a fluorophore having the following chemical structure:

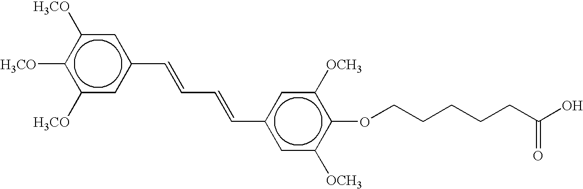

In an alternate embodiment, the present invention is a fluorophore having the following chemical structure:

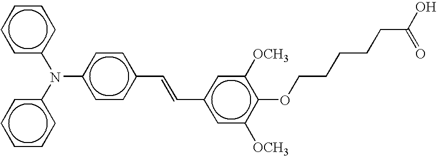

In an alternate embodiment, the present invention is a fluorophore having the following chemical structure:

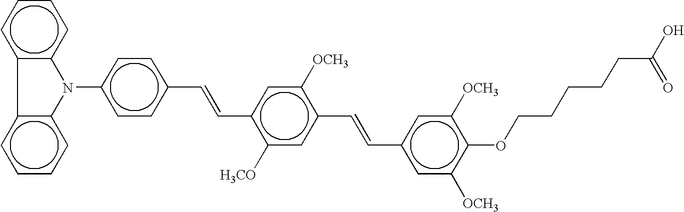

In an alternate embodiment, the present invention is a fluorophore having the following chemical structure:

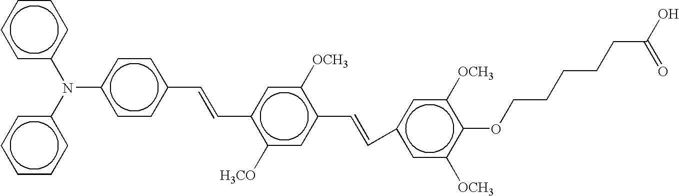

In an alternate embodiment, the present invention is a fluorophore having the following chemical structure.

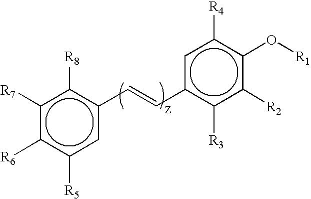

wherein R₁ is (CH₂)ₙX, wherein n=1-10 and X is selected from the group consisting of H, OH, SH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄, R₅, R₆, R₇ and R₉ are each selected from the group consisting of H, OH, CN, NO₂, NH₂, OCH₃, OCH₂CH₃, and (CH₂)ₙX, wherein n=1-0 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

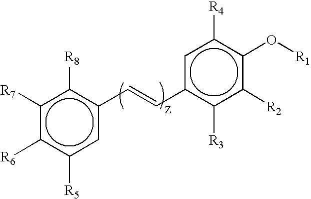

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

R₂, R₃, R₄, R₅, R₆, R₇ and R₈ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

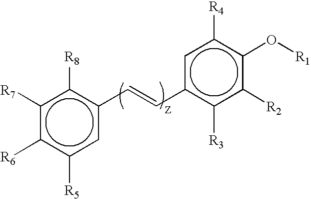

wherein R₁ is (CH₂CH₂O)ₘY, wherein m=1-20 and Y is selected from the group consisting of H, CH₃, CH₂CH₃, CH₂CH₂CH₃, CH₂H₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄, R₅, R₆, R₇ and R₉ are each selected from the group consisting of H, OH, CN, NO₂, NH₂, OCH₃, OCH₂CH₃, and (CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

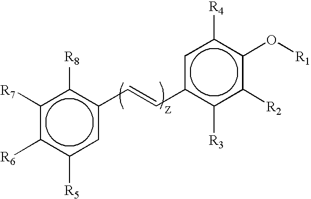

wherein R₁ is (CH₂CH₂O)ₘY, wherein m=1-20 and Y is selected from the group consisting of H, CH₃, CH₂CH₃, CH₂CH₂CH₃, CH₂H₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄, R₅, R₆, R₇ and R₈ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

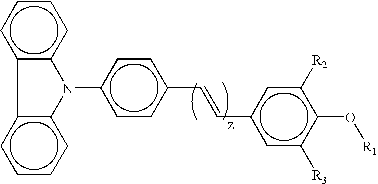

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂ and R₃ are each selected from the group consisting of H, OH, CN, NO₂—NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

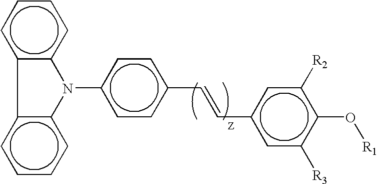

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂ and R₃ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

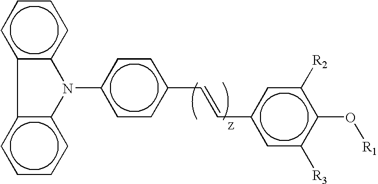

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂SH, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂ and R₃ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

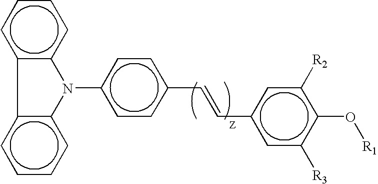

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂SH, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂ and R₃ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂ OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

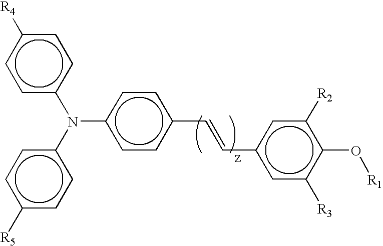

wherein R₁ is (CH₂)ₙX, wherein n=1-10 and X is selected from the group consisting of H, OH, SH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄ and R₅ are each selected from the group consisting of H, OH, SH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

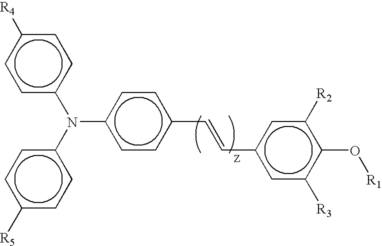

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

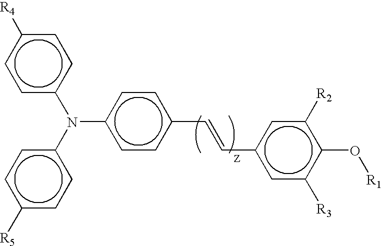

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂SH, CH₂CH₂CONH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄ and R₅ are each selected from the group consisting of H, OH, SH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

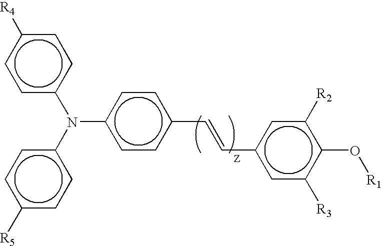

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂SH, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

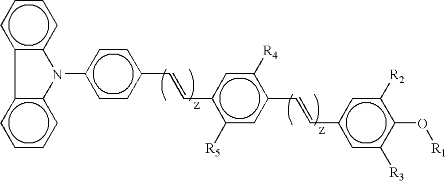

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄ and R₅ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

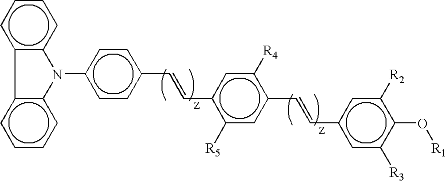

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

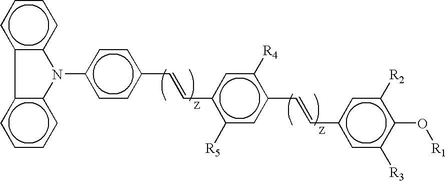

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂. Cl₂CH₂CO(N-hydroxysuccinimide), CO(N-hydroxysuccinimide), and CH₂CH₂NH(Biotin);

wherein R₂, R₃, R₄ and R₅ are each selected from the group consisting of U, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises on-e single bond and one double bond.

In an alternate embodiment, the present invention is a fluorophore having the following chemical structure:

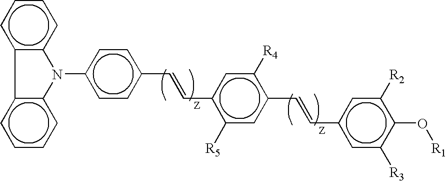

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure:

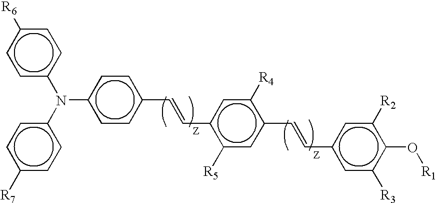

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄, R₅, R₆ and R₇ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

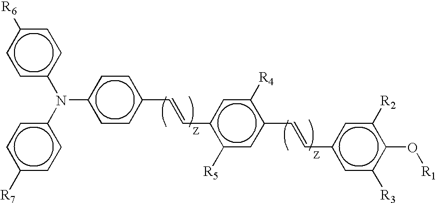

wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);

wherein R₂, R₃, R₄, R₅, R₆ and R₇ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

In an alternate embodiment, the present invention is a fluorophore having the following chemical structure;

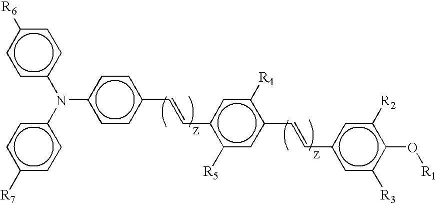

wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄, R₅, R₆ and R₇ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and

wherein Z is between one and five alternating pairs of single and double bonds, and wherein each pair of single and double bonds comprises one single bond and one double bond.

In an alternate embodiment, the present invention is a fluorophore having the following chemical structure:

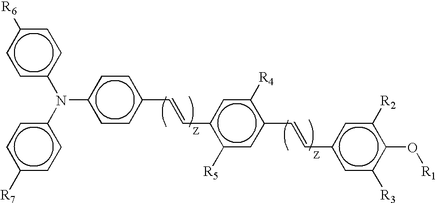

wherein R₁ is (CH₂CH₂O)ₘY, wherein m=1-20 and Y is selected from the group consisting of X, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);

wherein R₂, R₃, R₄, R₅, R₆ and R₇ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and

wherein Z is between one and five alternating pairs of single and double bonds, and wherein each pair of single and double bonds comprises one single bond and one double bond.

### BRIEF DESCRIPTION OF THE DRAWINGS

FIG. 1 is a diagram of certain synthesis steps that are common to all six compounds covered by the present invention.

FIG. 2 is a diagram of the steps involved in the synthesis of Compound 8A1 of the present invention.

FIG. 3 is a diagram of the steps involved in the synthesis of Compound 7B-cz of the present invention.

FIG. 4 is a diagram of the steps involved in the synthesis of Compound 8A2 of the present invention.

FIG. 5 is a diagram of the steps involved in the synthesis of Compound 7B of the present invention.

FIG. 6 is a diagram of the steps involved in the synthesis of Compound 7C-cz of the present invention.

FIG. 7 is a diagram of the steps involved in the synthesis of Compound 7C of the present invention.

FIG. 8 is a schematic representation of the chemical structure of Compound 8A1 of the present invention.

FIG. 9 is a schematic representation of the chemical structure of Compounds 8A1 and 8A2 of the present invention with annotations to facilitate the discussion of analogs of Compounds 8A1 and 8A2.

FIG. 10 is a schematic representation of the chemical structure of Compound 7B-cz of the present invention.

FIG. 11 is a schematic representation of the chemical structure of Compound 7B-cz of the present invention with annotations to facilitate the discussion of analogs of Compound 7B-cz.

FIG. 12 is a schematic representation of the chemical structure of Compound 8A2 of the present invention.

FIG. 13 is a schematic representation of the chemical structure of Compound 7B of the present invention.

FIG. 14 is a schematic representation of the chemical structure of Compound 7B of the present invention with annotations to facilitate the discussion of analogs of Compound 7B.

FIG. 15 is a schematic representation of the chemical structure of Compound 7C-cz of the present invention.

FIG. 16 is a schematic representation of the chemical structure of Compound 7C-cz of the present invention with annotations to facilitate the discussion of analogs of Compound 7C-cz.

FIG. 17 is a schematic representation of the chemical structure of Compound 7C of the present invention.

FIG. 18 is a schematic representation of the chemical structure of Compound 7C of the present invention with annotations to facilitate the discussion of analogs of Compound 7C.

FIG. 19 is a graph of the excitation spectra of the dyes of the present invention.

### DETAILED DESCRIPTION OF INVENTION

The present invention is a family of one- and two-photon dyes that have overlapping fluorescence excitation maxima between 365 nm and 425 nm and that are specifically functionalized for coupling to biomolecules. The dyes have defined Stokes shifts such that they produce a continuum of emission wavelengths. The dyes are based on a structure, diphenylamine bis stilbene, known to have good electron transfer and fluorescence characteristics (see U.S. Pat. No. 6,267,913 (Marder et al., 2001) and U.S. Patent Application Pub. No. 20020185634 (Marder et al.)). In contrast to the symmetrical bis stilbenes mentioned above, the dyes of the present invention are asymmetrical. The asymmetry of these dyes facilitates functionalization of the dyes with carboxyl, thiol, aldehyde-reactive or amine-reactive groups, which in turn facilitates coupling with macromolecules or functionalized microspheres. Furthermore, the dyes of the present invention are photostable and have high quantum yields (fluorescence intensity).

In a preferred embodiment, a single 370 nm source is used for excitation, and the specific emission spectrum of each dye produces a visual fluorescent color series when the beads are used as probes or the dyes are used to tag probe ligands in an assay. For two-photon applications, the structures of the dyes are preferably modified according to previously described prediction algorithms [2, 3] so that they have large two-photon cross-sections.

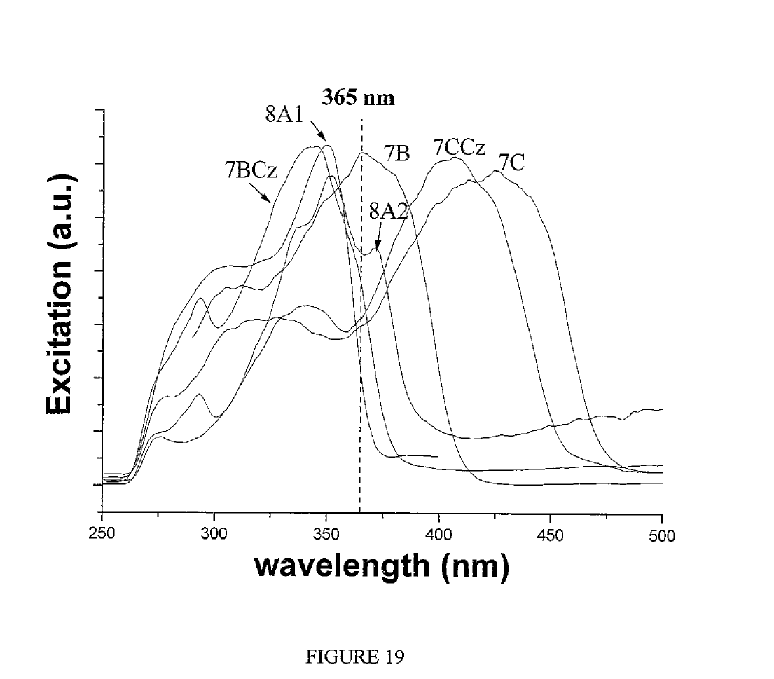

FIG. 19 is a graph of the excitation spectra of the dyes of the present invention.

1. Overview

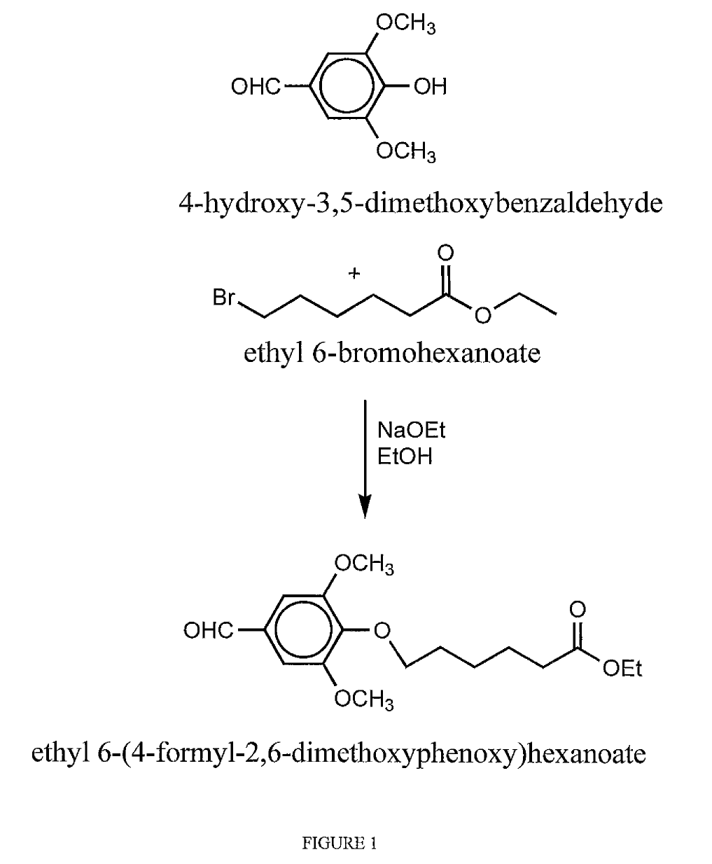

As illustrated in the examples below and set forth in Table 1, for lateral flow assays, the dyes of the present invention have spectra that overlap such that a single wavelength source, for example, a common black light at 365 nm, can be used for excitation (see FIG. 1). The fluorophores of the present invention have large Stokes shifts, and their emission spectra derived from a single excitation source have peaks (Em) that are spectroscopically separate. The effect is to provide a system for multiplex assays with the capacity to spectroscopically distinguish multiple analytes simultaneously. With proper choice of emission characteristics, at least five analytes coupled to individual dyes can be distinguished visually.

The fluorophores of the present invention are very photostable and have excellent quantum yields in nonpolar solvents and in aqueous environments when coupled to peptides and proteins, incorporated in polystyrene microspheres or coupled to dextrans. Potential applications for the fluorophores of the present invention include multiplex point-of-care diagnostics, antibody or substrate labeling, in vivo imaging, proteomics and genomics array techniques, flow cytometry, and use as dye taggants for security monitoring.

TABLE 1

| | | Molar | | | Stokes | QY |
|---|---|---|---|---|---|---|
| | λₘₐₓ | absorptivity | Ex | Em | shift | (Whilliams |
| Example | (nm) | (mM⁻¹cm⁻¹) | (nm) | (nm) | (Em-Ex) | method) |
| 1) 8A1 | 330 | 34 | 350 | 391 | 41 | 0.13 |
| 2) 7B-cz | 342 | 32 | 342 | 408 | 66 | 0.74 |
| 3) 8A2 | 352 | 49 | 351 | 430 | 79 | 0.26 |
| 4) 7B | 367 | 24 | 365 | 450 | 85 | 0.77 |
| 5) 7C-cz | 403 | 46 | 401 | 456 | 55 | 0.80 |
| 6) 7C | 414 | 53 | 425 | 500 | 75 | 0.78 |

*489 nm in DMF, 462 nm H₂O

Two-photon cross-sections for some of the above examples are: 7B-cz 430 GM@610 nm; 7B: 100 GM@600 nm; 7C-cz: 375 GM@675 nm; and 7C:1,000 GM @710 nm.

2. Synthesis Methods

FIG. 1 is a diagram of certain synthesis steps that are common to all six compounds covered by the present invention. These and all other steps involved in synthesizing the compounds of the present invention are discussed below.

### A. EXAMPLE 1

### Compound 8A1

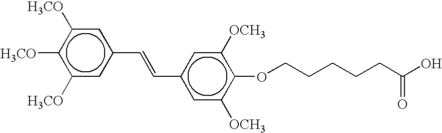

2 ml MeOH and 4 ml 20% NaOH were added to 5 ml tetrahydrofuran (THF) and cooled in an ice bath. 0.385 g (0.5 equivalents, 0.01019 mol) NaBH₄ were added slowly. This solution was then added to 4.0 g (0.02038 mol) of 3,4,5-trimethoxybenzaldehyde in a mix of 50 ml-1 THF and 50 ml MeOH and stirred at 20° C. for 8 hours. Most of the solvent was removed under vacuum and the remainder poured into 200 ml H₂O, neutralized with 2M HCl, extracted with 3×75 ml CH₂Cl₂, washed with 1×50 ml H₂O and 1×50 ml brine, and dried with MgSO₄. Removal of the solvent gave 3.88 g of oil, (3,4,5-trimethoxyphenyl)methanol, that was used in the next step without further purification. 3.8 g (0.01917 mol) of (3,4,5-trimethoxyphenyl)methanol were added to 150 ml dry Et₂O and cooled to 0° C. 0.90 ml (0.5 equivalents, 0.009585 mol) PBr₃ were added drop-wise and stirred for 12 hours at 20° C. The mixture was re-cooled and neutralized with NaHCO₃, extracted with 4×50 ml Et₂O, washed with 2×50 ml brine, and dried with MgSO₄ to yield 5-(bromoethyl)-1,2,3-trimethoxybenzene. 4.53 g (1.1 equivalents, 0.02242 mol) PBu₃ was added and stirred at 20° C. for 48 hours. The white precipitate, Wittig Salt I, was removed by vacuum filtration and washed with cold (10-15° C.) Et₂O to remove un-reacted PBu₃. Total yield of Wittig Salt I was 5.5 g or 58%.

2.0 g (0.006166 mol) of previously made ethyl 6-(4-formyl,-2,6-dimethoxyphenoxy)hexanoate and 3.57 g (1.25 equivalents, 0.007707 mol) of Wittig Salt I were added to 150 ml EtOH under N₂. 3.85 ml (1.25 equivalents, 0.007707 mol) 2M NaOEt was added drop-wise at 60° C. and stirred for 24 hours. The reaction was cooled, neutralized, and poured into 200 ml H₂O extracted with 4×50 ml CH₂Cl₂, washed with 2×50 ml H₂O and 1×50 ml brine, and dried with MgSO₄. The solvent was removed under vacuum and the residue, ethyl 6-(2,6-dimethoxy-4-((1E)-4-(3,4,5-trimethoxyphenyl)ethenyl)phenoxy)hexanoate, taken up in 75 ml 3M KOH, stirred at 60° C. for 6 hours, cooled, and re-acidified to pH 2.0 with 3M HCl. The mixture was extracted with 4×50 ml CH₂Cl₂, washed with 2×50 ml H₂O and 1×50 ml brine, and dried with MgSO₄. The solvent was removed, and the remaining material, product 8A1, 6-(2,6-dimethoxy-4-((1E)-4-(3,4,5-trimethoxyphenyl)ethenyl)phenoxy)hexanoic acid, was purified through a silica gel column, eluting with 70% CH₂Cl₂/30% EtOAc. Total yield of the final product, Compound 8A1, 6-(2,6-dimethoxy-4-((1E)-4-(3,4,5-trimethoxyphenyl)ethenylphenoxy)hexanoic acid, was 1.9 g or 67%.

¹H NMR (500 MHz, CDCl₃), δ 6.83 (m, 2H), 6.62 (m, 6H), 3.95 (t, J=7 Hz, 2H), 3.84 (s, 4H), 3.82 (s, 9H), 2.37 (t, J=7 Hz, 2H), 1.69 (m, 4H), 1.49 (m, 2H)

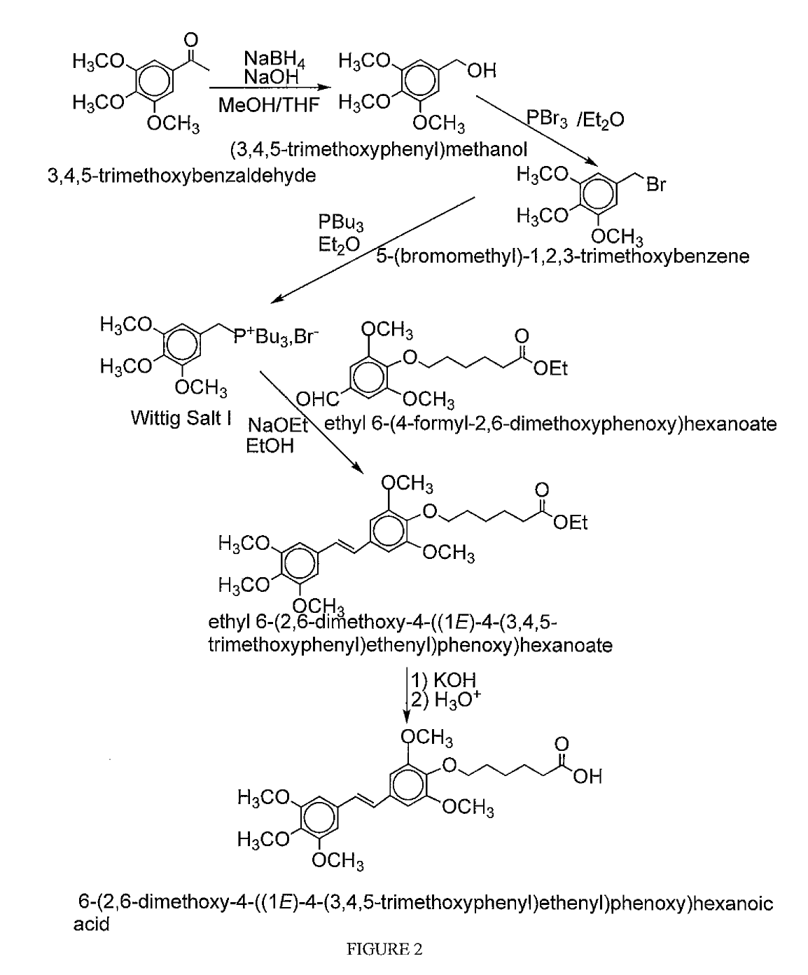

FIG. 2 is a diagram of the steps involved in the synthesis of Compound 8A1 of the present invention.

### B. EXAMPLE 2

### Compound 7B-cz

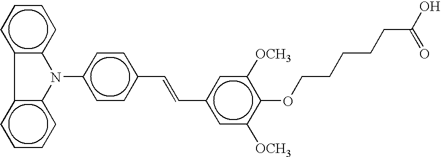

0.9 g (3.7 mol %) of P(t-Bu)₃ and 0.35 g (1.3 mol %) Pd(OAc)₂ were added to 800 ml degassed toluene, followed by 25 g (1.1 eq., 0.1316 mol) of 4-bromobenzaldehyde and 20 g (0.1196 mol) of 9H-carbazole and 41.5 g (2.5 eq., 0.2990 mol) anhydrous K₂CO₃. The reaction mixture was heated to 115° C. for 24 hours. Most of the solvent was removed and the reaction stirred for another 12 hours at 115° C. Solvent was removed under vacuum, and 300 ml CH₂Cl₂ was added. The material was washed with 2×50 ml H₂O and 2×50 ml 20% NaOH, washed with 1×50 ml brine, and dried with MgSO₄. The solvent was removed, leaving the intermediate, 4-(9H-carbazol-9-yl)benzaldehyde, an orange-brown solid. 125 ml of 80% hexanes/20% EtOAc were added. The mixture was stirred, cooled and vacuum filtered, leaving an off-white solid. After washing with more hexanes/EtOAc, the white solid was recrystallized from ethanol/toluene to give 20.53 g fine white needle crystals. A second crop of crystals provided 4.34 additional grams of the intermediate, 4-(9H-carbazol-9-yl)benzaldehyde. Total yield was 24.87 g or 76.6%.

¹H NMR (500 MHz, CDCl₃), δ 10.10 (s, 1H), 8.13 (dd, J=8.5 Hz), 7.78 (d, J=8.5 Hz, 2H), 7.49 (d, J=8 Hz, 2H), 7.43 (dd, J=8.5 Hz, 2H), 7.32 (dd, J=8.5 Hz, 2H)

4.3 g (0.1585 mol) of the intermediate, 4-(9H-carbazol-9-yl)benzaldehyde, 0.3 g (0.5 equivalents, 0.007925 mol) NaBH₄, and 2.3 ml 20% NaOH were added to a mixture of 75 ml THF and 75 ml MeOH. The solution was stirred for 16 hours at 20° C. Most of the solvent was removed under vacuum and the remainder poured into 200 ml H₂O. The mixture was neutralized with 3M HCl, extracted with 3×50 ml CH₂Cl₂, and washed with 2×40 ml H₂O and 1×40 ml brine. The solvent was removed under vacuum, and the residue was taken up in 20 ml hot CH₂Cl₂. Hexanes were added drop-wise until the desired intermediate, (4-(9H-carbazol-9-yl)phenyl)methanol, precipitated out. 3.58 g of fine needles of the alcohol product were obtained. The total yield of intermediate, (4-(9H-carbazol-9-yl)phenyl)methanol, was 3 was 3.58 g or 83%.

3.5 g (0.01281 mol) of the intermediate, (4-(9H-carbazol-9-yl)phenyl)methanol, was added to a mixture of 75 ml dry Et₂O and 10 ml dry THF under N₂ and cooled to 0° C. 0.6 ml (0.5 equivalents, 0.006405 mol) PBr₃ was added drop-wise and then stirred for 16 hours at 20° C. The mixture was re-cooled and neutralized with NaHCO₃, extracted with 3×50 ml Et₂O, washed with 2×50 ml brine, and dried with MgSO₄. The solvent was reduced to ˜75 ml under vacuum, and 3.36 g (1.0 equivalent, 0.01281 mol) PBu₃ was added to the intermediate, 9-(4-(bromomethyl)phenyl)-9H-carbazole, and stirred for 48 hours. The resulting white precipitate was removed by vacuum filtration and washed with cold (0-15° C.) Et₂O to remove unreacted PBu₃. 4.35 g of a white powder, Wittig Salt II, was obtained. The total yield was 4.35 g or 74%.

4.0 g of previously made ethyl 6-(4-formyl-2,6-dimethoxyphenoxy)hexanoate (0.01233 mol) and 8.3 g of Wittig Salt II (1.25 equivalents, 0.01541 mol) were added to 150 ml 95% EtOH under N₂. The mixture was heated to 60° C., and 7.8 ml 2M NaOEt (1.25 equivalents, 0.01541 mol) was added drop-wise. The mixture was stirred at 60° C. for 20 hours. The mixture was then cooled to room temperature and neutralized, and 250 ml H₂O was added. The resulting mixture was extracted with 4×50 ml CH₂Cl₂, washed with 1×50 ml H₂O and 1×50 ml brine, and dried with MgSO₄. The solvent was removed under vacuum, and 50 ml 3M KOH was added to the residue, ethyl 6-(4-(4-(9H-carbazol-9-yl)styryl)-2,6-dimethoxyphenoxy)hexanoate. The mixture was stirred for 4 hours at 70° C., cooled, re-acidified with 3M HCl to pH 3.0, extracted with 4×50 ml CH₂Cl₂, washed with 1×50 ml H₂O and 1×50 ml brine, and dried with MgSO₄. Removal of solvent gave a brown oil, 6-(4-(4-(9H-carbazol-9-yl)styryl)-2-,6-dimethoxyphenoxy)hexanoic acid or Compound 7B-cz, which was purified through a silica gel column, eluting with 80% CH₂Cl₂/20% EtOAc. The total yield of final product, 6-(4-(4-(9H-carbazol-9-yl)styryl)-2,6-dimethoxyphenoxy)hexanoic acid, Compound 7B-cz, was 4.25 g or 65%.

¹H NMR (500 MHz, CDCl₃), δ 8.13 (d, J=7.5 Hz, 2H), 7.70 (d, J=8.5 Hz, 2H), 7.54 (d, J=8.0 Hz, 2H), 7.415 (m, 4H), 7.28 (m, 2H), 7.10 (s, 2H), 6.77 (s, 2H), 3.91 (s, 6H), 3.98 (t, J=7 Hz, 2H), 2.32 (t, J=7 Hz, 2H), 1.72 (m, 4H), 1.53 (m, 2H)

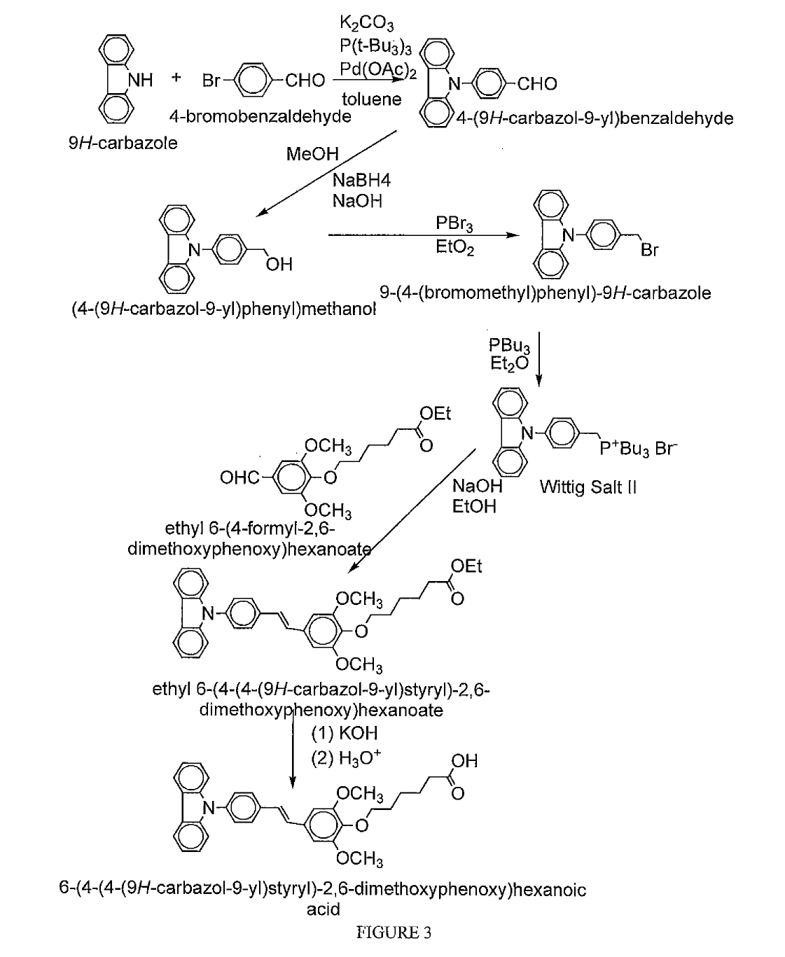

FIG. 3 is a diagram of the steps involved in the synthesis of Compound 7B-cz of the present invention.

### C. EXAMPLE 3

### Compound 8A2

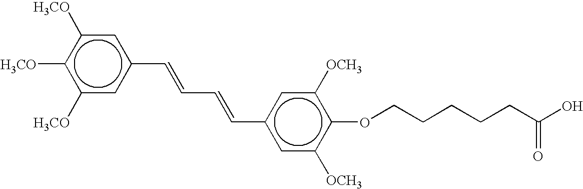

5.0 g (0.02548 mol) of 3,4,5-trimethoxy benzaldehyde and 33 ml of a 1M solution of previously made Wittig Salt III [4] in dimethylformamide (DMF) (1.3 equivalents, 0.03312 mol) was added to 150 ml 95% EtOH under N₂ and warmed to dissolve all solids. 19 ml 2M NaOEt (1.5 equivalents, 0.03822 mol) was added drop-wise, and the reaction was heated to 60° C. for 48 hours. The mixture was poured into 300 ml H₂O, extracted with 3×75 ml EtOAc, washed with 2×40 ml H₂O and 2×40 ml brine, dried with MgSO₄, and concentrated by rotary evaporation. The resulting yellow solids were added to 150 ml THF. 100 ml 2M HCl was added to this solution and stirred for one hour at 20° C. Most of the solvent was removed by rotary evaporation, and the remainder was poured into 100 ml H₂O. The product was extracted, washed, and dried as before, giving 5.2 g of impure yellow oil. This oil was purified through a short silica gel column, eluting with CH₂Cl₂ giving 4.02 g of a yellow solid, (E)-4-(3,4,5-trimethoxyphenyl)but-3-en-2-one intermediate product. The total yield was 4.02 g or 71%.

¹H NMR (500 MHz, CDCl₃), δ 9.66 (d, J=8 Hz, 1H), 7.37 (d, J=16 Hz, 1H), 6.77 (s, 2H), 6.62 (dd, J=16 Hz, J=8 Hz, 1H), 3.88 (s, 9H)

0.73 g LAH (0.35 equivalents, 0.01811 mol) were added to 200 ml dry THF at 0° C. 11.5 g (0.05175 mol) of the (E)-4-(3,4,5-trimethoxyphenyl)but-3-en-2-one intermediate product in 100 ml THF were added drop-wise. The mixture was stirred at 20° C. for 6 hours, cooled to 0° C., and 50 ml of cold (10-15° C.) H₂O were slowly added to the mixture. The solution was acidified to pH 5.0 and extracted with 4×50 ml CH₂Cl₂, washed with 2×50 ml H₂O, and dried with MgSO₄. The solvent was removed under vacuum. The (E)-3-(3,4,5-trimethoxyphenyl)prop-2-en-1-ol intermediate product was purified through a silica gel column, eluting with 80% CH₂Cl₂/20% hexanes 80% CH₂Cl₂/20% EtOAc. The total yield of the (E)-4-(3,4,5-trimethoxyphenyl)prop-2-en-1-ol intermediate product was 10.7 g or 93%.

3.5 g (0.01561 mol) of the (E)-4-(3,4,5-trimethoxyphenyl)prop-2-en-1-ol intermediate product were added to a mixture of 100 ml dry Et₂O and 50 ml dry THF. The flask was placed under N₂ and cooled to 0° C. 2.11 g (0.5 equivalents, 0.007805 mol) PBr₃ were added drop-wise, and the flask was stirred for 18 hours while allowing the temperature to increase to 20° C. The solution was re-cooled to 0° C. and poured into 100 ml cold (10-15° C.) saturated NaHCO₃. An additional 50 ml Et₂O was added, and the mixture was washed with 1×50 ml H₂O and 2×50 ml brine to yield 5((E)-3-bromoprop-1-enyl)-1,2,3-trimethoxybenzens, used directly in the next step. The solvent mixture was dried with MgSO₄ and then placed under N₂. 3.90 g (1.0 equivalents, 0.01561 mol) of PBu₃ were added drop-wise, and the reaction was stirred at 20° C. for 48 hours. The resulting Wittig Salt IV was removed by vacuum filtration and washed twice with Et₂O to remove any unreacted PBu₃. The resulting white solid was recrystallized from hexanes/isopropyl alcohol (IPA) to give 5.50 g of a white powder, Wittig Salt IV. The total yield of Wittig Salt IV was 5.50 g or 72%.

¹H NMR (500 MHz, CDCl₃), 6, 7.36 (d, J=16 Hz, 1H), 6.77 (s, 2H), 6.62 (dd, J=16 Hz, J=8 Hz, 1H), 3.88 (s, 9H), 1.44 (m, 12H), 2.62 (d, J=16 Hz, 2H), 2.38 (t, J=11 Hz, 6H), 0.94 (t, J=11 Hz, 9H)

2.90 g (0.008940 mol) of previously made ethyl 6-(4-form-yl-2,6-dimethoxyphenoxy)hexanoate and 5.47 g (1.25 equivalents, 0.01117 mol) of Wittig Salt IV were added to a mixture of 125 ml EtOH and 25 ml DMF under N₂ and cooled to 0° C. 5.6 ml (1.25 equivalents, 0.01117 mol) of 2M NaOEt were added drop-wise. The temperature was allowed to rise to 2000. The mixture was stirred for 12 hours and then heated to 70° C. for 6 hours. The solution was poured into 300 ml H₂O and neutralized with 2M HCl. The solution was extracted with 4×75 ml CH₂Cl₂, washed with 2×50 ml H₂O and 1×50 ml brine, dried with MgSO₄, and concentrated by rotary evaporation. The resulting ester intermediate, ethyl6-(2,6-dimethoxy-4-(1E,3E)-4-(3,4,5-trimethoxyphenyl)buta-1,3-dienyl)phenoxy)hexanoate, was then converted to the acid by stirring in 75 ml 2.5M KOH at 60° C. for 4 hours, cooling to 0° C., and then re-acidifying with 6M HCl to pH 3.0. The reaction mixture was extracted with 4×75 ml CH₂Cl₂, washed with 2×50 ml 1420 and 1×50 ml brine, dried with MgSO₄, and concentrated by rotary evaporation. The final product, 6-(2,6-dimethoxy-4-(1E,3E)-4-(3,4,5-trimethoxyphenyl)buta-1,3-dienyl)phenoxy)hexanoic acid, Compound 8A2, was purified through a silica gel column, eluting with 50% EtOAc/50% hexanes, and then recrystallized from EtOAc/1-exanes to give 2.82 g of yellow crystals. The total yield of the final product, 6-(2,6-dimethoxy-4-(1E,3E)-4-(3,4,5-trimethoxyphenyl)buta-1,3-dienyl)phenoxy)hexanoic acid, Compound 8A2, was 2.82 g or 65%.

¹H NMR (500 MHz, CDCl₃), δ 6.81 (m, 2H), 6.64 (s, 2H), 6.63 (s, 2H), 6.56 (m, 2H), 3.96 (t, J=7 Hz, 2H), 3.86 (s, 15H), 2.37 (t, J=7 Hz, 2H), 1.73 (m, 4H), 1.52 (m, 2H)

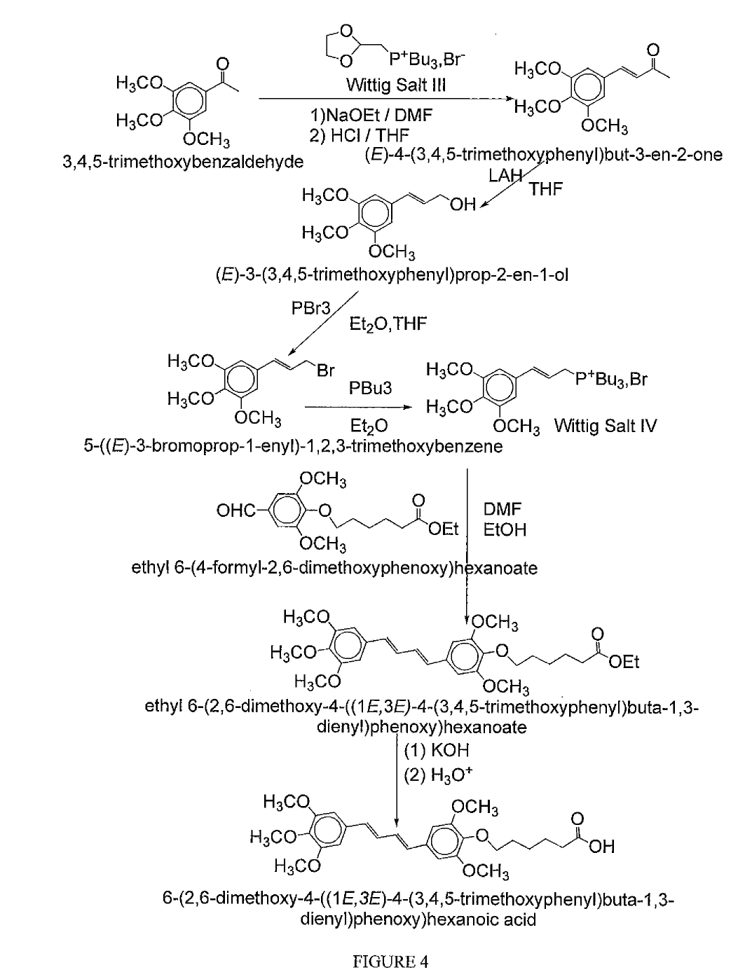

FIG. 4 is a diagram of the steps involved in the synthesis of Compound 8A2 of the present invention.

### D. EXAMPLE 4

### Compound 7B

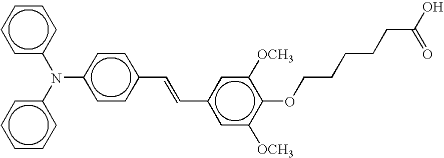

25.0 g (0.102 mol) triphenylamine and 16 ml (2.0 equivalents, 0.204 mol) of DMF were added to ˜150 ml of CHCl₃ and cooled to 0° C. 16.0 ml (1.5 equivalents, 0.153 mol) of POCl₃ were added drop-wise. The flask was allowed to slowly warm to 20° C., stirred for 12 hours, and then refluxed for 2 hours. The solution was cooled and poured into 250 ml cold water (10-15° C.) and then neutralized with aqueous Na₂CO₃. The solution was extracted with 4×75 ml CH₂Cl₂, washed with 3×50 ml H₂O, dried with MgSO₄, and then concentrated by rotary evaporation. The resulting yellow solid, 4-(diphenylamino)benzaldehyde, was recrystallized from EtOH, giving 20.08 g of bright yellow crystals. A second crop of crystals from the mother liquor gave an additional 5.10 g of 4-(diphenylamino)benzaldehyde. The total yield was 25.18 g or 90.4%.

20.0 g (0.07317 mol) of 4-(diphenylamino)benzaldehyde were added to a mixture of 100 ml THF and 100 ml of MeOH. 1.38 g (0.5 equivalents, 0.365 mol) NaBH₄ and 15 ml of 20% aqueous NaOH were added to this solution. The solution was stirred at 20° C. for 12 hours. Most of the solvent was removed by rotary evaporation. The remainder was poured into 300 ml H₂O, extracted with 4×75 ml CH₂Cl₂, washed with 2×50 ml H₂O, and dried with MgSO₄. After removal of the solvent, the resulting solid was recrystallized from EtOH to give a white solid, (4-(diphenylamino)phenylmethanol. The total yield was 18.5 g or 92%.

32.0 g (0.0592 mol) of the (4-(diphenylamino)phenyl)methanol were dissolved in a mixture of 150 ml of dry Et₂O and 150 ml dry THF. The flask was placed under N₂ and cooled to 0° C. 5.6 ml of PBr₃ (0.5 equivalents, 0.0296 mol) were added drop-wise, and the flask was allowed to slowly warm to 20° C. while stirring for 12 hours. Approximately half of the solvent was removed by rotary evaporation. The remainder was poured into 300 ml of H₂O, then extracted with 3×100 ml Et₂O and washed with 2×50 ml H₂O and 1×50 ml brine. The intermediate product, N-(4-(bromomethyl)phenyl)-N-phenylbenzamide, was dried with MgSO₄ and filtered into a 500 ml round bottom flask. The flask was placed under N₂, and 14.8 ml (1.0 equivalent, 0.0592 mol) of PBu₃ were added drop-wise and stirred at 20° C. for 48 hours. The resulting Wittig salt, Witting Salt V, was removed by vacuum filtration and washed several times with Et₂O to remove any unreacted PBu₃. The product was pure enough to use directly but can be recrystallized from EtOAc/EtOH if necessary. The intermediate product, Wittig Salt V, was obtained. The total yield was 24.9 g or 78%.

4.0 g of previously made ethyl 6-(4-formyl-2,6-dimethoxyphenoxy)hexanoate (0.01233 mol) and 8.66 g (1.3 equivalents, 0.01603 mol) of Wittig Salt V were added to approximately 150 ml degassed EtOH under N₂ and cooled to 0° C. 7.8 mL (1.25 equivalents, 0.01541 mol) 2M NaOEt was added drop-wise. The temperature was allowed to slowly rise to 20° C., and the mixture was stirred for 16 hours. It was then heated to reflux for 4 hours. Most of the solvent was removed by rotary evaporation, and the remainder was poured into 200 ml H₂O. The solution was neutralized with 2M HCl, extracted with 4×75 ml CH₂Cl₂, washed with 2×50 ml H₂O and 1×50 ml brine, dried with MgSO₄, and concentrated by rotary evaporation. The resulting intermediate product, ethyl 6-(4-(4-(diphenylamino)styryl)-2,6-dimethoxyphenoxy)hexanoate, was then converted to the acid by stirring in 75 ml of 2.5 M KOH at 60° C. for 4 hours, cooling to 0° C., and then re-acidifying with 6 M HCl to pH 3.0. The resulting final product was extracted with 4×75 ml CH₂Cl₂, washed with 2×50 ml brine, dried with MgSO₄, and concentrated by rotary evaporation. The final product was purified through a medium-length, large-diameter silica gel column, eluting with 50% EtOAc/50% hexanes. It was then recrystallized from EtOAc/hexanes to give a yellow solid, 6-(4-(4-(diphenylamino)styryl)-2,6-dimethoxyphenoxy)hexanoic acid, Compound 7B. The total yield of Compound 7B was 3.85 g or 58%.

¹H NMR (500 MHz, CDCl₃), δ 7.35 (d, J=8.5 Hz, 2H), 7.24 (dd, J=8.5 Hz, 4H), 7.10 (2, J=8.5 Hz, 2H), 7.02 (m, 4H), 6.91 (m, 2H), 6.69 (s, 2H), 3.96 (t, J=7 Hz, 2H), 3.87 (s, 6H), 2.39 (t, J=7 Hz, 2H), 1.80 (m, 2H) 1.70 (m, 2H), 1.51 (m, 2H)

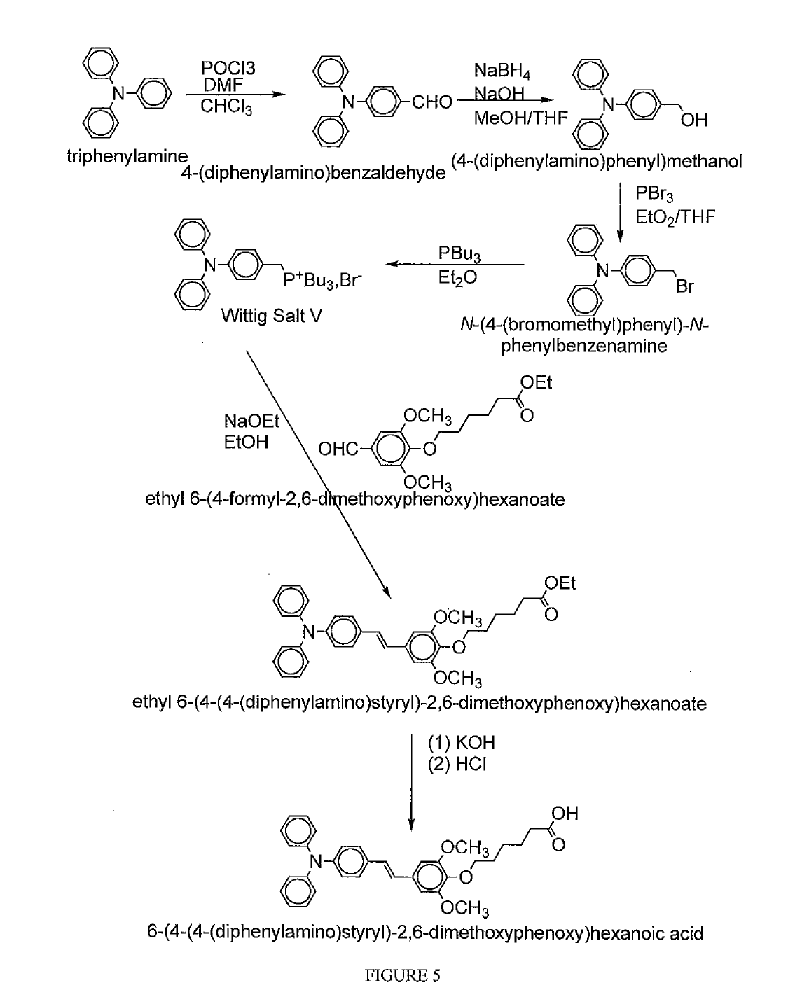

FIG. 5 is a diagram of the steps involved in the synthesis of Compound 7B of the present invention.

### E. EXAMPLE 5

### Compound 7C-cz

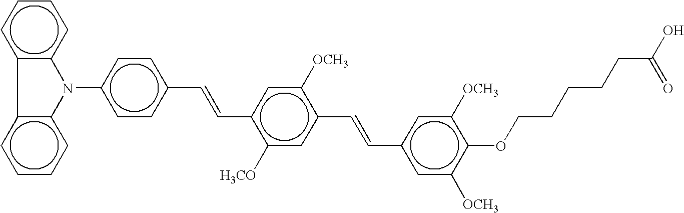

0.9 g (3.7 mol %) of P(t-Bu)₃ and 0.35 g (1.3 mol %) Pd(OAc)₂ were added to 800 ml degassed toluene, followed by 25 g (1.1 eq., 0.1316 mol) of 4-bromobenzaldehyde, 20 g (0.1196 mol) of 9H-carbazole, and 41.5 g (2.5 eq., 0.2990 mol) anhydrous K₂CO₃. The reaction mixture was heated to 115° C. for 24 hours. Most of the solvent was removed under vacuum and the reaction stirred for another 12 hours at 115° C. The remaining solvent was removed under vacuum, and 300 ml CH₂Cl₂ was added to the reaction mixture. The material was washed with 2×50 ml H₂O and 2×50 ml 20% NaOH, then with 1×50 ml brine, and then dried with MgSO₄. The solvent was removed, leaving an orange-brown solid. 125 ml of 80% hexanes/20% EtOAc was added to the mixture, which was stirred, cooled and vacuum filtered, leaving an off-white solid. After washing with more hexanes/EtOAc, the white solid was recrystallized from ethanol/toluene to give 20.53 g fine white needle crystals of 4-(9H-carbazol-9-yl)benzaldehyde intermediate product. A second crop of crystals provided 4.34 additional grams of the 4-(9H-carbazol-9-yl)benzaldehyde intermediate product. The total yield was 24.87 g or 76.6%.

¹H NMR (500 MHz, CDCl₃), δ 10.10 (s, 1H), 8.13 (dd, J=8.5 Hz), 7.78 (d, J=8.5 Hz, 2H), 7.49 (d, J=8 Hz, 2H), 7.43 (dd, J=8.5 Hz, 2H), 7.32 (dd, J=8.5 Hz, 2H)

4.0 g (0.01474 mol) of the 4(9H-carbazole-9-yl)benzaldehyde and 14.1 g (1.5 eq., 0.02211 mol) of Wittig Salt VI [5] were added to a mixture of 150 ml dry THF and 250 ml IPA under N₂. The solution was cooled to 0° C., and 16.5 ml (1.1 eq., 0.01621 mol) of 1 M t-Bu3O⁻K⁺ were added drop-wise. The temperature was allowed to rise slowly to 20° C., and the reaction was stirred for 6 hours. After removing most of the solvent, 250 ml CH₂Cl₂ were added. The solution was washed with 3×50 ml H₂O and 1×50 ml brine, dried with MgSO₄, and evaporated, leaving a thick, yellow slurry that was purified through a short silica gel column eluting with CH₂Cl₂ followed by 85% CH₂Cl₂/15% MeOH. The total yield of the intermediate product, Wittig Salt VII, was 6.25 g or 62%.

¹H NMR (500 MHz, CDCl₃), δ 8.13 (d, J=7.5 Hz, 2H), 7.76 (d, J=7.5 Hz, 2H), 7.55-7.04 (m, 12H), 4.10 (d, J=24 Hz, 2H), 3.90 (s, 6H), 2.40 (t, J=11 Hz, 6H), 1.44 (m, 12H), 0.90 (t, J=11 Hz, 9H)

5.24 g (0.007985 mol) of Wittig Salt VII and 2.34 g (0.9 equivalents, 0.007186 mol) of previously made ethyl 6-(4-formyl-2,6-dimethoxyphenoxy)hexanoate were added to a mix of 1100 ml 95% EtOH/50 ml IPA and stirred under N₂. 5 ml of 2M NaOH (1.25 equivalents, 0.009981 mol) were added drop-wise. The mixture was stirred at 20° C. for 48 hours and poured into 300 ml H₂O. The resulting aqueous mixture was extracted with 4×50 ml CH₂Cl₂ washed with 1×50 ml H₂O and 1×50 ml brine, and dried with MgSO₄. The solvent was removed under vacuum, leaving the ester intermediate ethyl6-(4-(4-(4-(9H-carbazol-9-yl)styryl)-2,5-dimethoxystyryl)-2,6-dimethoxyphenoxy)hexanoate, and 50 ml 3M KOH were added. The mixture was stirred at 70° C. for 4 hours and then cooled and acidified to pH 3.0, extracted with 4×50 ml CH₂Cl₂, washed with 1×50 ml H₂O and 1×50 ml brine, and dried with MgSO₄. The solvent was removed under vacuum, and the resulting final product, 6-(4-(4-(4-(9H-carbazol-9-yl)styryl-2,5-dimethoxystyryl)-dimethoxyphenoxy)hexanoic, Compound 7C-cz, was purified through a silica gel column, eluting with a gradient of CH₂Cl₂/hexanes CH₂Cl₂/EtOAc. The total yield of 6-(4-(4-(4-(9H-carbazol-9-yl)styryl-2,5-dimethoxystyryl)-dimethoxyphenoxy)hexanoic, Compound 7C-cz was 3.65 g or 66%.

¹H NMR (500 MHz, CDCl₃), δ 8.13 (d, J=7.5 Hz, 2H), 7.76 (d, J=7.5 Hz, 2H), 7.55-7.04 (m, 14H), 6.76 (s, 2H), 3.98 (t, J=7 Hz, 2H), 3.89 (s, 12H), 2.32 (t, J=7 Hz, 2H), 1.72 (m, 4H), 1.53 (m, 2H)

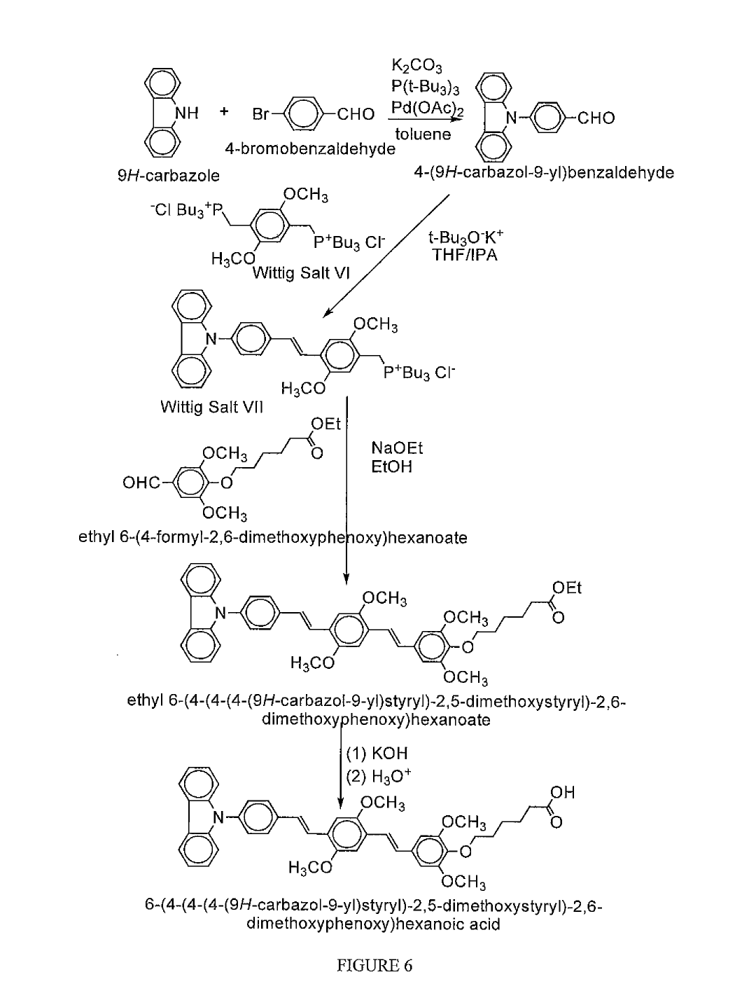

FIG. 6 is a diagram of the steps involved in the synthesis of Compound 7C-cz of the present invention.

### F. EXAMPLE 6

### Compound 7C

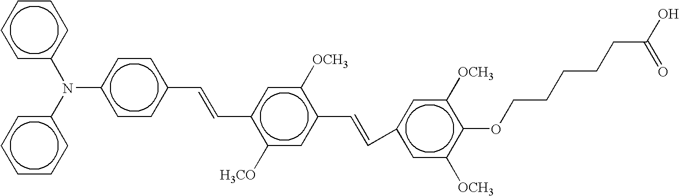

25.0 g (0.102 mol) triphenylamine and 16 mL (2.0 equivalents, 0.204 mol) of DMF were added to ˜150 ml of CHCl₃ and cooled to 0° C. 16.0 ml (1.5 equivalents, 0.153 mol) of POCl₃ was added drop-wise. The flask was allowed to slowly warm to 20° C., and the mixture was stirred for 12 hours and then refluxed for 2 hours. The solution was cooled and poured into 250 ml cold water (10-15° C.) and then neutralized with aqueous Na₂CO₃. The solution was extracted with 4×75 ml CH₂Cl₂, washed with 3×50 ml H₂O, dried with MgSO₄, then concentrated by rotary evaporation. The resulting yellow solid, 4-(diphenylamino)benzaldehyde intermediate, was recrystallized from EtOH, giving 20.08 g of bright yellow crystals. A second crop of crystals from the mother liquor gave an additional 5.10 g of product. The total yield of 4-(diphenylamino)benzaldehyde was 25.18 g or 90.4%.

2.0 g of the 4-(dithenylamino)benzaldehyde intermediate (0.007317 mol) and 7.0 g (1.5 equivalents, 0.01098 mol) of Wittig Salt VI [5] were added to a mixture of 75 ml THF and 75 ml IPA. The flask was placed under N₂ and cooled to 0° C. 8.4 ml (1.1 equivalents, 0.008049 mol) of 1 M t-Bu₃O⁻K⁺ were added drop-wise. The flask was allowed to warm to 20° C. and stirred for 4 hours. Most of the solvent was removed by rotary evaporation, and the remainder was poured into 200 ml water, extracted with 4×75 ml CH₂Cl₂, washed with 3×50 ml H₂O, and dried with MgSO₄. The impure yellow oil was purified through a short-length, large-diameter silica gel column, eluting with CH₂Cl₂/15% MeOH. The intermediate product, Wittig Salt VIII, consisted of yellow oil. The total yield of Wittig Salt VIII was 3.2 g or 65%.

¹H NMR (500 MHz, CDCl₃), δ 7.35 (d, J=8.5 Hz, 4H), 7.24 (d, J=16 Hz, 1H), 7.09 (d, J=8 Hz, 6H), 7.02 (m, 5H), 6.72 (s, 1H), 6.44 (s, 1H), 4.11 (d, J=24 Hz, 2H), 3.81 (s, 6H), 2.40 (t, J=11 Hz, 6H), 1.44 (m, 12H), 0.90 (t, J=11 Hz, 9H)

1.60 g (0.004923 mol) of previously made ethyl 6-(4-formyl-2,6-dimethoxyphenoxy)hexanoate and 4.0 g (1.25 eq., 0.006165 mol) of Wittig Salt VIII were added to 150 ml degassed 95% ethanol under N₂. The reaction flask was cooled to 0° C., and 3.20 ml (1.25 eq., 0.006165 mol) 2M NaOEt was added drop-wise. The reaction mixture was allowed warm to 20° C. and stirred for 18 hours, then heated to reflux for 4 hours. The mixture was concentrated by rotary evaporation, giving an impure yellow-green liquid that was purified through a short-length, large-diameter silica gel column, eluting with 90% CH₂Cl₂/10% hexanes, leaving 3.8 g semi-pure ethyl 6-(4-(4-(4-(diphenylamino)styryl)-2,5-dimethoxystyryl)-2,6-dimethoxyphenoxy)hexanoate, a yellow-orange oil, after removal of the solvent. The oil was added to 100 ml 2.5 M KOH and 50 ml IPA, stirred at 60° C. for 6 hours, cooled to 0° C., and acidified to pH 3.0 with 3 M HCl. An additional 200 ml H₂O was added to the reaction mixture, and the mixture was extracted with 4×75 ml CH₂Cl₂, washed with 2×50 ml H₂O containing a trace of HCl, and then washed with 1×50 ml brine. The solution was dried with MgSO₄ and concentrated by rotary evaporation. The product, a red-orange oil, was purified through a short-length, large-diameter silica gel column eluting with 60% EtOAc/40% hexanes to give a yellow solid final product, 6-(4-(4-(4-(diphenylamino)styryl)-2) 2,5-dimethoxystyryl)-2,6-dimethoxyphenoxy)hexanoic acid, Compound 7C. The total yield of Compound 7C was 2.58 g or 66%.

¹H NMR (500 MHz, CDCl₃), δ 7.40 (d, J=8.5 Hz, 2H), 7.34 (m, 2H), 7.24 (m, 6H), 7.10 (m, 4H), 7.03 (m, 61H), 6.70 (m, 2H), 3.96 (t, J=7 Hz, 2H), 2.39 (t, J=7 Hz, 2H), 1.80 (m, 2H) 1.70 (m, 2H), 1.51 (m, 2H)

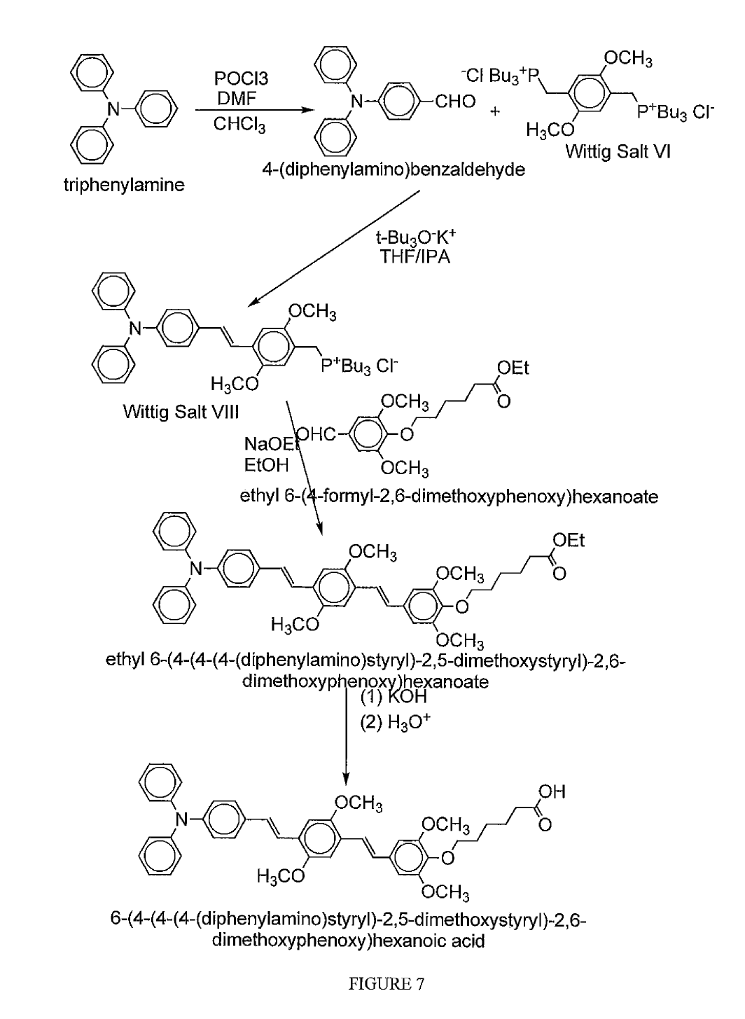

FIG. 7 is a diagram of the steps involved in the synthesis of Compound 7C of the present invention.

3. Structures and Analogs

A. Structure and Analogs of Compound 8A1

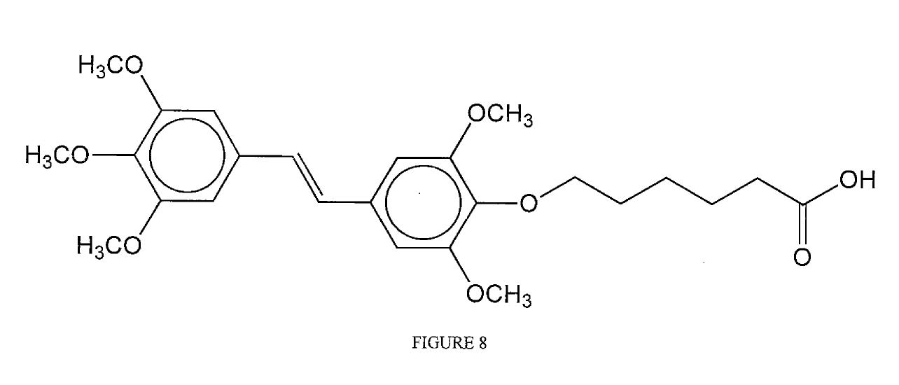

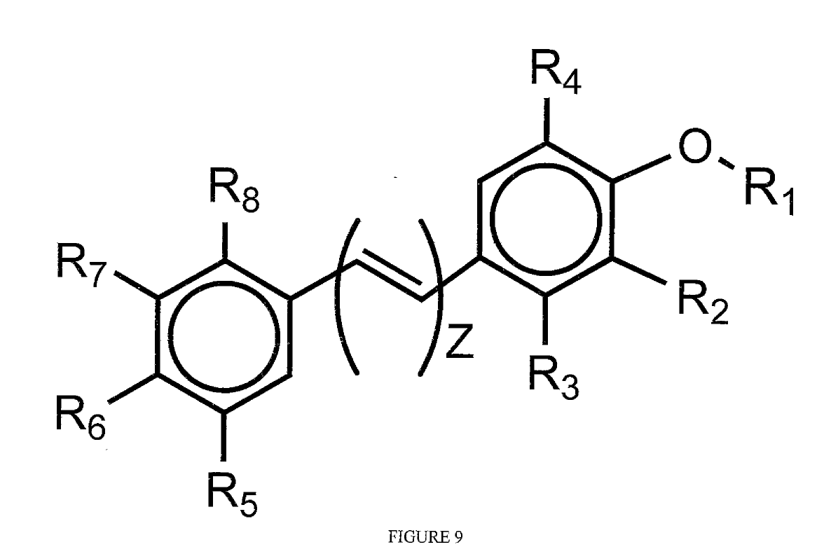

FIG. 8 is a schematic representation of the chemical structure of Compound 8A1 of the present invention. Referring to FIG. 9, in one embodiment of the present invention, R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₘY, wherein m=1-20 and Y is selected from the group consisting of H, CH₃, CH₂CH₃, CH₂CH₂CH₃, CH₂H₂COOH, CH₂CH₂SH, CH₂CH₂COOCH₃, CH₂CH₂CONHNH₂, CH₂CH₂CONH₂, CH₂CH₂CO N-hydroxysuccinimide), CO(N-hydroxysuccinimide), and CH₂CH₂NH(Biotin).

In one embodiment of the present invention, R₂, R₃, R₄, R₅, R₆, R₇ and R₈ are each selected from the group consisting of H, OH, SH, CN, NO₂, NH₂, OCH₃, OCH₂CH₃, and (CH₂)ₙX, wherein n=1-10 and X is selected from the group consisting of H, OH, SH, CN, NO₂, NH₂, COOHN, COCH₃, and COCH₂CH₃. In an alternate embodiment, R₂, R₃, R₄, R₅, R₆, R₇ and R₈ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

B. Structure and Analogs of Compound 7B-cz

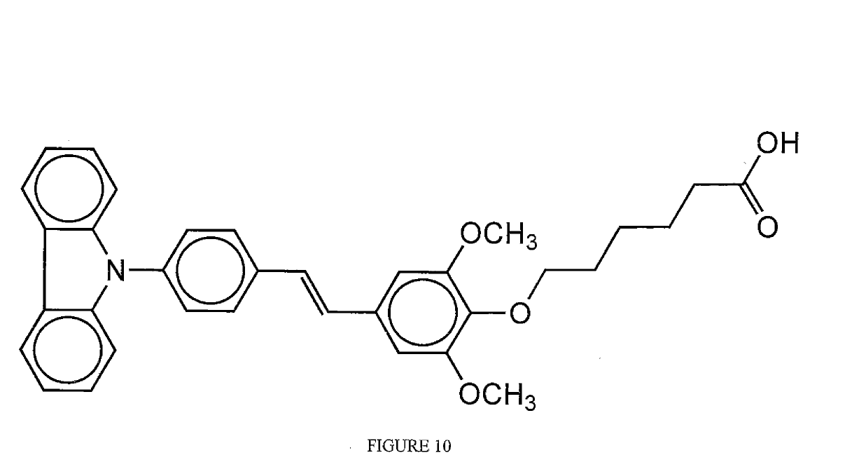

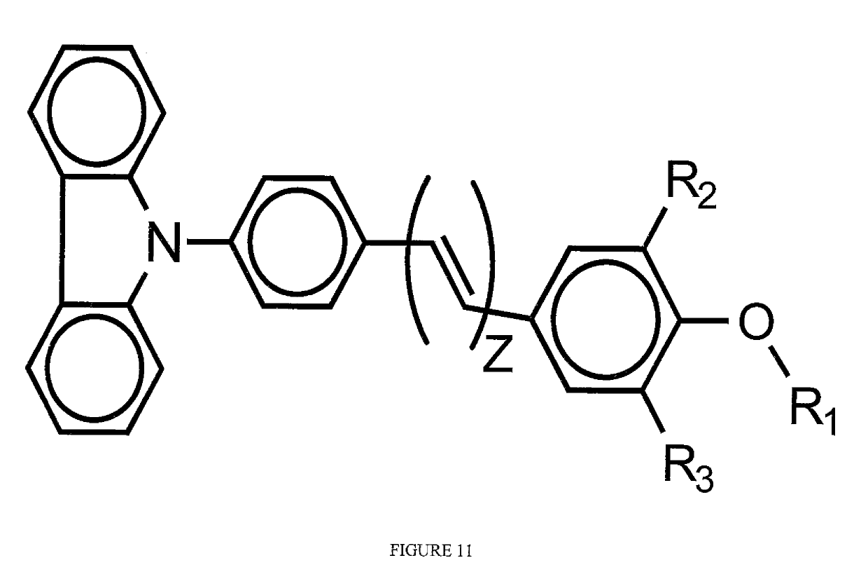

FIG. 10 is a schematic representation of the chemical structure of Compound 7B-cz of the present invention. Referring to FIG. 11, in one embodiment of the present invention, R₁ is (CH₂)ₙX, wherein n=10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₘY, wherein m=1-20 and Y is selected from the group consisting of H₃CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin).

In one embodiment of the present invention, R₂ and R₃ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃. In an alternate embodiment, R₂ and R₃ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

C. Structure and Analogs of Compound 8A2

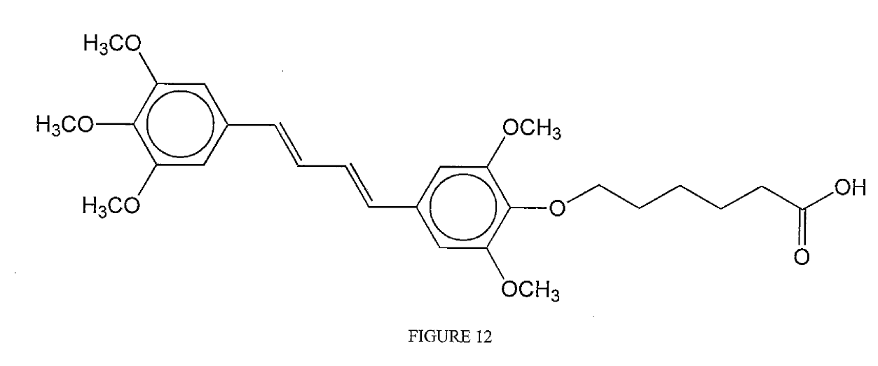

FIG. 12 is a schematic representation of the chemical structure of Compound 8A2 of the present invention. Referring to FIG. 9, in one embodiment of the present invention, in one embodiment of the present invention, R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₙY, wherein m=1-20 and Y is selected from the group consisting of H, CH₃, CH₂CH₃, CH₂CH₂CH₃, CH₂H₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin).

In one embodiment of the present invention, R₂, R₃, R₄, R₅, R₆, R₇ and R₅ are each selected from the group consisting of H, OH, CN, NO₂, NH₂, OCH₃, OCH₂CH₃, and (CH₂)ₙX, wherein n=1-10 and X is selected from the group consisting of H, OH, SH, CN, NO₂, NH₂, COOH, COCH₃, and COCH₂CH₃. In an alternate embodiment, R₂, R₃, R₄, R₅, R₆, R₇ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

Note that Compounds 8A1 and 8A2 are analogs of one another.

D. Structure and Analogs of Compound 7B

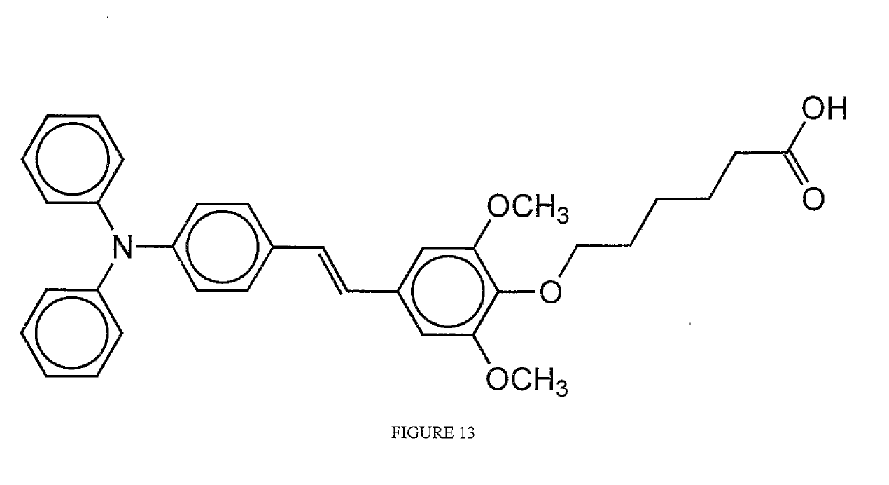

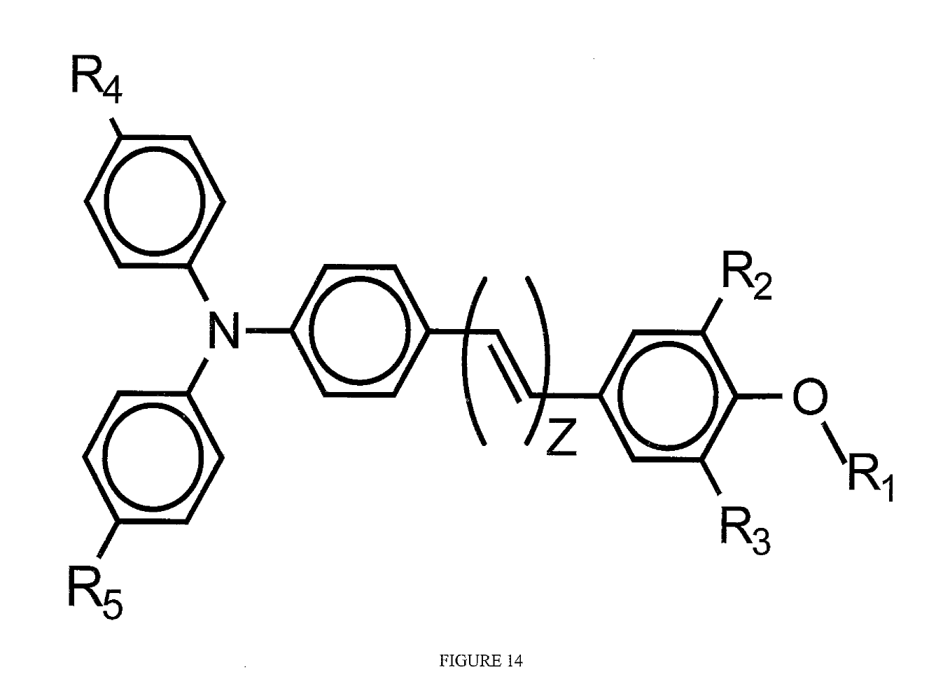

FIG. 13 is a schematic representation of the chemical structure of Compound 7B of the present invention. Referring to FIG. 14, in one embodiment of the present invention, R₁ is (CH₂)ₙX, wherein n=1-10 and X is selected from the group consisting of H, OH, SH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin).

In one embodiment of the present invention, R₂, R₃, R₄ and R₅ are each selected from the group consisting of H, OH, CN, NO₂, NH₁₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃. In an alternate embodiment, R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

E. Structure and Analogs of Compound 7C-cz

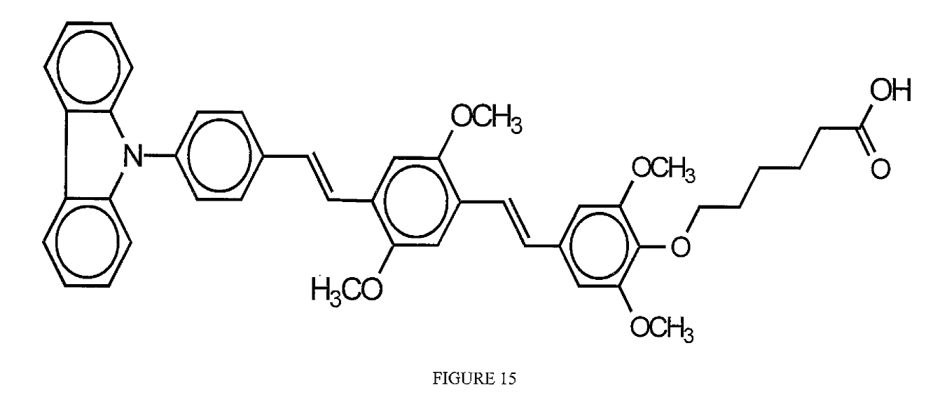

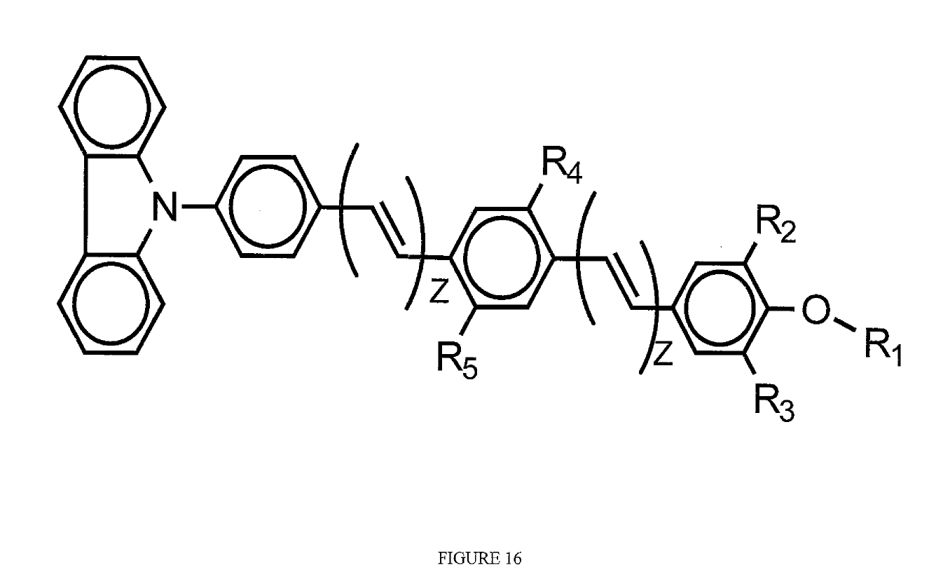

FIG. 15 is a schematic representation of the chemical structure of Compound 7C-cz of the present invention. Referring to FIG. 16, in one embodiment of the present invention, R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin).

In one embodiment of the present invention, R₂, R₃, R₄ and R₅ are each selected from the group consisting of H, OH, CN, NO₂, NH₂, OCH₃, OCH₂CH₃, and O(CH₂), wherein n=1-10 and X is selected from the group consisting of H, OH, SH, CN, NO₂, NH₂, COOH, COCH₃, and COCH₂CH₃. In an alternate embodiment, R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

F. Structure and Analogs of Compound 7C

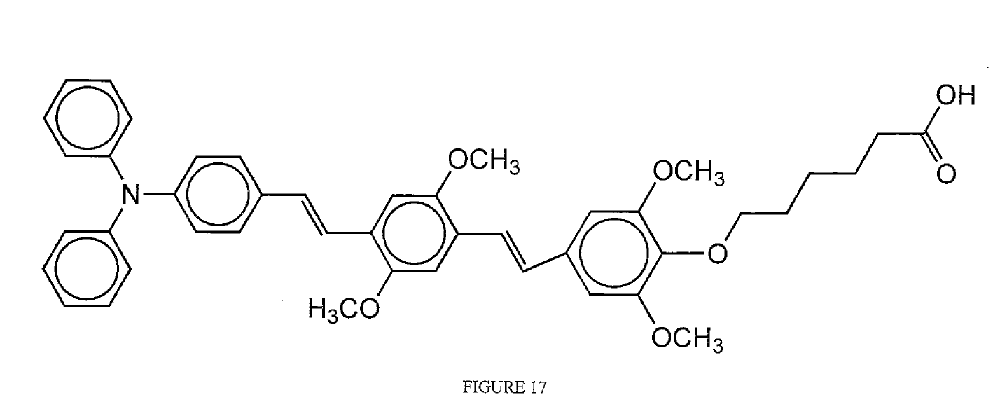

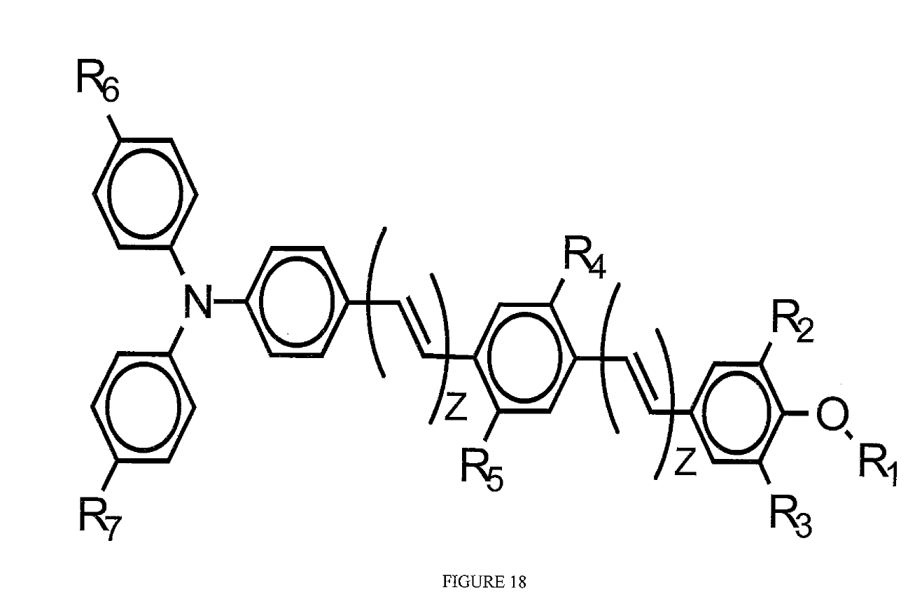

FIG. 17 is a schematic representation of the chemical structure of Compound 7C of the present invention. Referring to FIG. 18, in one embodiment of the present invention, R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin). In an alternate embodiment, R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin).

In one embodiment of the present invention, R₂, R₃, R₄, R₅, R₆ and R₇ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃. In an alternate embodiment, R₂, R₃, R₄, R₅, R₆ and R₇ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin).

In a preferred embodiment, Z represents conjugated double bonds, and there are between one and five double bonds connected in sequence so that there can be one, two, three, four, or five consecutive double bonds connected via single bonds so as to make a conjugated sequence of alternating single and double bonds. In other words, Z represents alternating single and double bonds, and there are between one and five pairs of single and double bonds, i.e., single-double (one pair), single-double-single-double (two pairs), single-double-single-double-single-double (three pairs), single-double-single-double-single-double-single-double (four pairs), or single-double-single-double-single-double-single-double-single-double (five pairs).

4. Practical Applications

Fluorophores containing the functionalized spacers described in the above examples (i.e., an alkane linker chain terminating in a reactive functional groups such as a carboxylate, hydrazide or N-hydroxysuccinimide ester) can be coupled to suitably functionalized microspheres for use in lateral flow assay applications. Fluorophores containing an alkane-capped spacer (i.e., an alkane linker chain terminating in a methyl group) can be infused into microspheres for similar types of lateral flow assays. In either case, the result is fluorescent microspheres that can be coupled to biomolecular ligands such as antibodies, receptor ligands and receptor analogs for use in point-of-care diagnostic tests, either multiplexed or single analysis lateral flow assays.

Point-of-care diagnostics using microspheres impregnated with fluorophores include tests for bioweapons, infectious diseases, food and water pathogens, drugs of abuse, biomedical research, pregnancy and fertility testing, assays for tumor markers, rheumatic diseases, and gasteroenteric disease markers to identify celiac or Crohns disease or agricultural and veterinary diseases. Recent data comparing commercial-style gold nanoparticle-based lateral flow assays to fluorescent microspheres impregnated with Compound 7C showed that use of the 7C fluorescent microspheres in an Influenza A lateral flow assay resulted in at least a ten-fold quantitative enhancement in sensitivity over a comparable gold nanoparticle-based assay for Influenza A [6]. Additional uses include coupling fluorescent microspheres to specific receptor ligands or other biomolecules for flow cytometry assays and cell sorting applications.

The fluorophores of the present invention can be coupled directly to biomolecules or small organic molecules via the functional groups appended to the alkane or poly(ethyleneglycol) spacers (i.e., an alkane or poly(ethyleneglycol) linker chain terminating in a reactive functional group such as a carboxylate, hydrazide or N-hydroxysuccinimide ester). In this configuration the fluorophores are suitable for: imaging applications when coupled to dextrans; for receptor binding and enzyme tracing applications; for neurophysiology, for example, when coupled to cholera toxin for retrograde labeling of neurological pathways; functional studies of enzyme-substrate interactions; labeling of primary or secondary antibodies in Enzyme-linked Immunosorbant Assays (ELISA); confocal microscopy; use as a taggant for motor oils; use as a label for currency security; use as a label for multiplexed PCR and gene sequencing; and Forster Resonance Energy Transfer (FRET)-based assays of enzyme function and DNA hybridization.

The above discussion includes some of the many potential uses of the fluorophores of the present invention but is not intended to provide an exhaustive list, nor is it intended to limit the scope of the present invention in any respect.

Although several preferred embodiments of the present invention have been shown and described, it will be apparent to those skilled in the art that many changes and modifications may be made, or variations utilized, without departing from the invention in its broader aspects. The appended claims are therefore intended to cover all such changes, modifications and variations as fall within the true spirit and scope of the invention.

### REFERENCES

1. Lakowicz, J. R. Principles of Fluorescence Spectroscopy, 3^{rd} ed. (2006). Ch. I. Introduction to Fluorescence Spectroscopy 1-25. Springer Science+Business Media. New York.
2. Drobizhev, M., A. Rebane, C. Sigel, E. Elandaloussi and C. W. A. Rebane, C. Sigel, E. H. Elandaloussi, and C. W. Spangler (2000). Picosecond dynamics of excitations studied in three generations of new 4,4′-bis(diphenylamino)stilbene-based dendrimers. Chem. Phys. Lett. 325: 375-82.
3. Drobizhev, M., A. Karotki, A. Rebane, and C. W. Spangler (2001). Dendrimer molecules with record large two-photon absorption cross section. Opt. Lett. 26: 1081-83.
4. Spangler, C. W. and R. K. McCoy (1988). Preparation of Conjugated Aromatic Polyenals by Wittig Oxypropenylation. Synthe. Commun. 18(1): 51-59.
5. Havelka, K. O. (1991). Stabilization of Polaronic and Bipolaronic Charge States in Electroactive Oligomers and Polymers. Doctoral Dissertation, Northern Illinois University, DeKalb, Ill.
6. Bauer, J. (Jul. 17 and 18, 2007). Oral presentation, American Association of Clinical Chemistry. San Diego.

We claim:

1. A fluorophore having the following chemical structure:

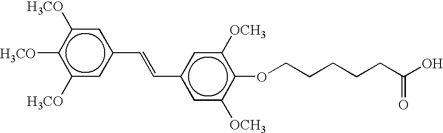

2. A fluorophore having the following chemical structure:

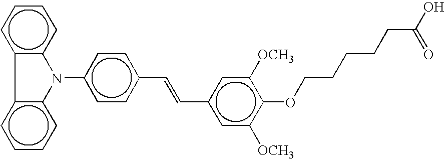

3. A fluorophore having the following chemical structure:

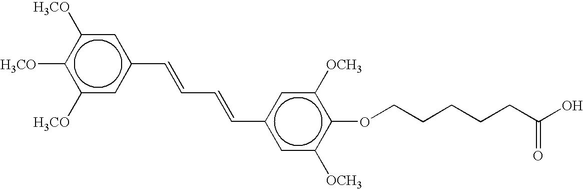

4. A fluorophore having the following chemical structure:

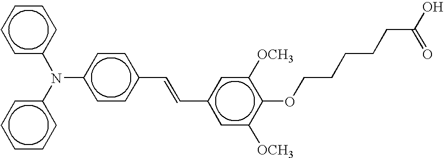

5. A fluorophore having the following chemical structure:

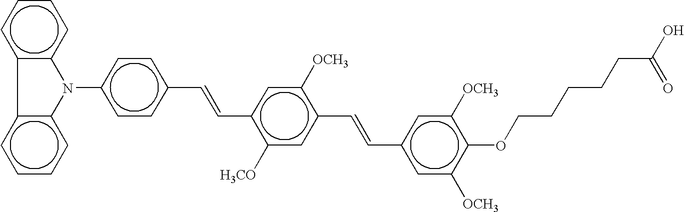

6. A fluorophore having the following chemical structure:

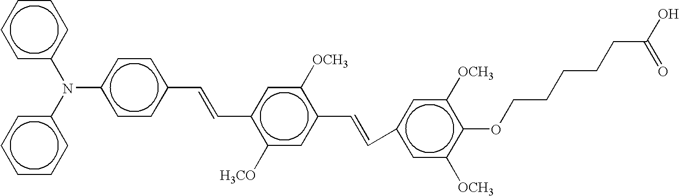

7. A fluorophore having the following chemical structure: 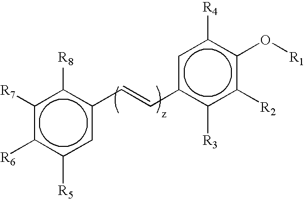
wherein R₁ is (CH₂)ₙX, wherein n=1-10 and X is selected from the group consisting of H, OH, SH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄, R₅, R₆, R₇ and R₈ are each selected from the group consisting of H, OH, CN, NO₂, NH₂, OCH₃, OCH₂CH₃, and (CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

8. A fluorophore having the following chemical structure: 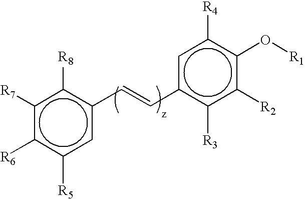
wherein R₁ is (CH₂)ₙX, wherein n=1-10 and X is selected from the group consisting of H, OH, SH, CO OH, COCH₃, COCH₂CH₃, CO(N-hydroxysuccinimide), OCO(N-hydroxysuccinimide), NH₂, NHNH₂, SH, and NH(Biotin);
R₂, R₃, R₄, R₅, R₆, R₇ and R₈ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-1-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

9. A fluorophore having the following chemical structure: 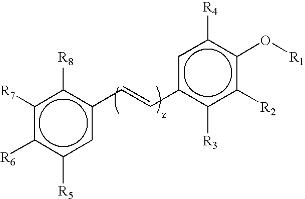
wherein R₁ is (CH₂CH₂O)ₘY, wherein m=1-20 and Y is selected from the group consisting of H, CH₃, CH₂CH₁, CH₂CH₂CH₃, CH₂H₂COOH, CH₂CH₂SH, CH₂CH₂COOCH₃,CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂, R₃, R₄, R₅, R₆, R₇ and R₈ are each selected from the group consisting of H, OH, CN, NO₂, NH₂, OCH₃, OCH₂CH₃, and (CH₂)ₙX, wherein n=1-10 and X is selected from the group consisting of H, OH, SH, CN, NO₂ NH₂, COOH, COCH₃, and COCH₂CH₃; and
wherein Z is between one and five alternating pairs of single and double bonds, and wherein each pair of single and double bonds comprises one single bond and one double bond.

10. A fluorophore having the following chemical structure: 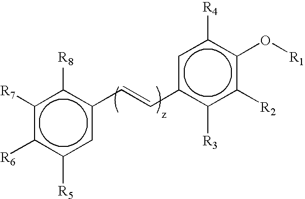
wherein R₁ is (CH₂CH₂O)ₘY, wherein m=1-20 and Y is selected from the group consisting of H, CH₃, CH₂CH₃, CH₂CH₂CH₃, CH₂H₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂, R₃, R₄, R₅, R₆, R₁ and R₈ are each (OCH₂CH₂)ₘY, wherein m=1-20 and Y is selected from the group consisting of OH, SH, OCH₃, OCH₂CH₃, OCH₂CH₂CH₃. OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

11. A fluorophore having the following chemical structure: 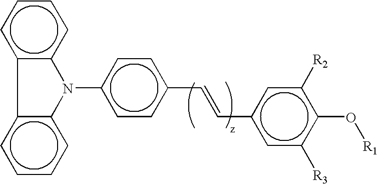
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccimide), NH₂, NHNH₂, SH, and NH(Biotin);
wherein R₂ and R₃ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

12. A fluorophore having the following chemical structure: 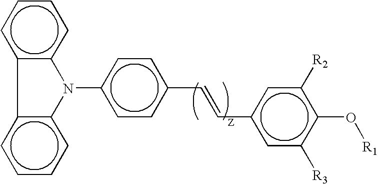
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);
wherein R₂ and R₃ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

13. A fluorophore having the following chemical structure: 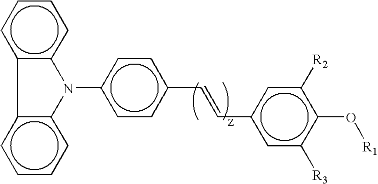
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂ and R₃ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

14. A fluorophore having the following chemical structure: 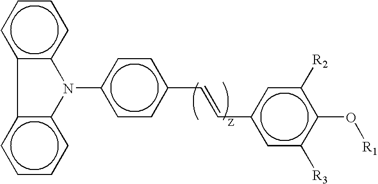
wherein R₁ is (CH₂CH₂O)ₘY, wherein m=1-20 and Y is selected from the group consisting of H, CH₃, CH₂CH₃, CH₂CH₂CH₃, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂ and R₃ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

15. A fluorophore having the following chemical structure: 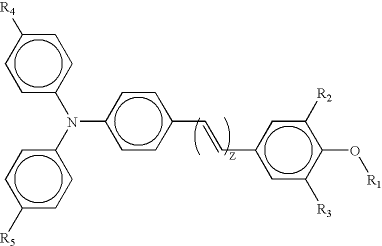
wherein R₁ is (CH₂)ₙX, wherein n=1-10 and X is selected from the group consisting of H, OH, SH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄ and R₅ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

16. A fluorophore having the following chemical structure: 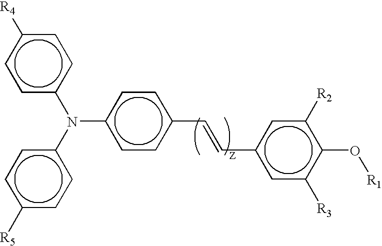
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, 1× and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

17. A fluorophore having the following chemical structure: 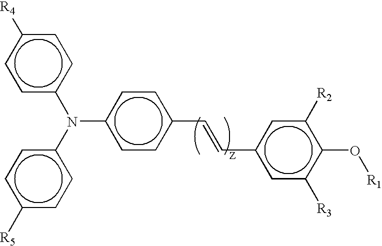
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂Cl₂CO(N-hydroxysuccinimide), CO(N-hydroxysuccinimide), and CH₂CH₂NH(Biotin);
wherein R₂, R₃, 4 and R₅ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

18. A fluorophore having the following chemical structure: 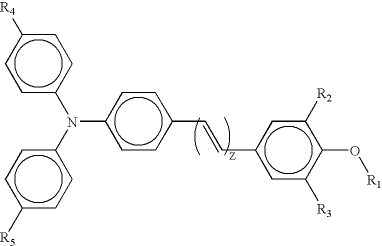
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂, R₃, R₄ and R₅ are each (OCH₂CH₂)ₘY, wherein m=1-20 and Y is selected from the group consisting of OH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

19. A fluorophore having the following chemical structure: 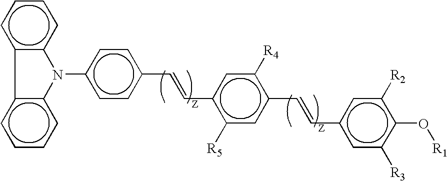
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄ and R₅ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

20. A fluorophore having the following chemical structure: 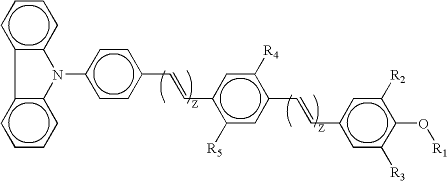
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄ and R₅ are each (OCH₂CH₂)ₙY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

21. A fluorophore having the following chemical structure: 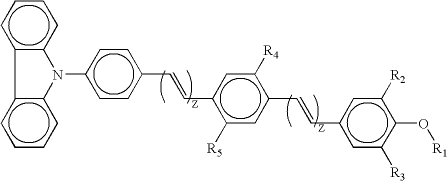
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂, R₃, R₄ and R₅ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

22. A fluorophore having the following chemical structure 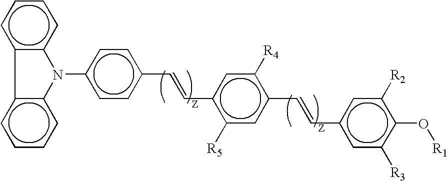
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccimide), CO(N-hydroxysuccinimide), and CH₂CH₂NH(Biotin);
wherein R₂, R₃, R₄ and R₅ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

23. A fluorophore having the following chemical structure: 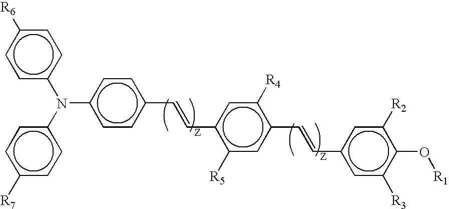
wherein R₁ is (CH₂)ₙX, wherein n=1-10 and X is selected from the group consisting of H, OH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄, R₅, R₆ and R₇ are each selected from the group consisting of H, OH, CN, NO₂, NH₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

24. A fluorophore having the following chemical structure: 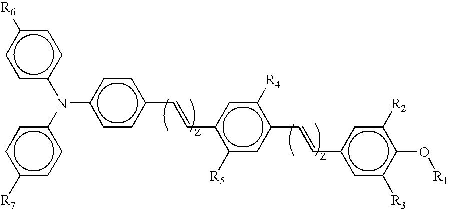
wherein R₁ is (CH₂)ₙX, wherein n=1-10 and X is selected from the group consisting of 1H, OH, COOH, COCH₃, COCH₂CH₃, CO(N-hydroxysuccinimide), OCO(N-hydroxysuccinimide), NH₂, NHNH₂, SH, and NH(Biotin);
wherein R₂, R₃, R₄, R₅, R₆ and R₇ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃ OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.

25. A fluorophore having the following chemical structure: 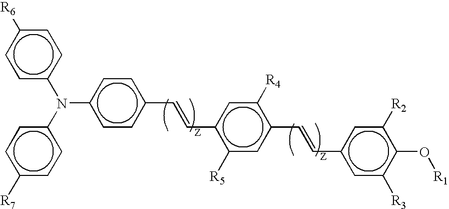
wherein R₁ is (CH₂CH₂O)ₘY, wherein m=1-20 and Y is selected from the group consisting of H, CH₃, CH₂CH₃, CH₂CH₂CH3, CH₂CH₂COOHN, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂CH₂NH(Biotin);
wherein R₂, R₃, R₄, R₅, R₆ and R₇ are each selected from the group consisting of H, OH, CN, NO₂, NH₁₂, OCH₃, OCH₂CH₃, and O(CH₂)ₙX, wherein n=1-10 and X is selected from the group consisting of H, OH, SH, CN, NO₂, NH₂, COOH, COCH₃, and COCH₂CH₃; and
wherein Z is between one and five alternating pairs of single and double bonds, and wherein each pair of single and double bonds comprises one single bond and one double bond.

26. A fluorophore having the following chemical structure: 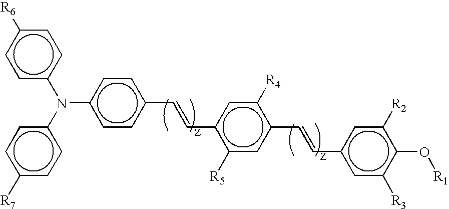
wherein R₁ is (CH₂CH₂O)ₘY, wherein m=1-20 and Y is selected from the group consisting of H, CH₃, CH₂CH₃, CH₂CH₂CH3, CH₂CH₂COOH, CH₂CH₂SH, CH₂CH₂COOCH₃, CH₂CH₂CONHNH₂, CH₂CH₂CONH₂, CH₂CH₂CO(N-hydroxysuccinimide), CO(N-hydroxysuccinimide), and CH₂H₂CH₂NH(Biotin);
wherein R₂, R₃, R₄, R₅, R₆ and R₇ are each (OCH₂CH₂)ₘY, wherein m=1-20 and Y is selected from the group consisting of OH, SH, OCH₃, OCH₂CH₃, OCH₂CH₂CH₃, OCH₂CH₂COOH, OCH₂CH₂COOCH₃, OCH₂CH₂CONHNH₂, OCH₂CH₂CONH₂, OCH₂CH₂CO(N-hydroxysuccinimide), OCO(N-hydroxysuccinimide), and OCH₂CH₂NH(Biotin); and
wherein Z is between one and five alternating pairs of single and double bonds, and wherein each pair of single and double bonds comprises one single bond and one double bond.
